(19) United States
(12) Patent Application Publication
(10) Pub. No.: US 2022/0254498 A1
(43) Pub. Date: Aug. 11, 2022

(54) DETECTING APPARATUS AND DETECTING METHOD

(71) Applicant: MAXELL, LTD., Kyoto (JP)

(72) Inventors: Yuko SANO, Tokyo (JP); Akihiko KANDORI, Tokyo (JP)

(21) Appl. No.: 17/628,598

(22) PCT Filed: May 11, 2020
(86) PCT No.: PCT/JP2020/018855

§ 371 Date: Jan. 20, 2022

(30) Foreign Application Priority Data
Jul. 22, 2019 (JP) 2019-134881

(51) Int. Cl. G16H 50/30

(52) U.S. Cl. CPC G16H 50/30

(57) ABSTRACT

In the cyclic time-series data anomaly-part detecting system 1, the overall-data evaluating unit 13 detects the anomaly of the overall data 44A based on the overall-data feature amount 44B, and besides, creates the feature-amount importance level 45B. The anomaly-part detecting system 1 creates the partial data 46A by dividing the overall data 44A that is the cyclic time-series data, calculates the partial-data feature amount 46B, and displays and outputs the partial-data anomaly detection result 46C that is the detection result of the anomaly of the partial data 46A, based on the partial-data feature amount 46B and the feature-amount importance level 45B.

### TECHNICAL FIELD

The present invention relates to an information processing service technique. And, the present invention relates to a technique for achieving a detecting apparatus and a detecting method detecting an anomaly part of cyclic time-series data.

### BACKGROUND ART

In a health care field, a medical field, a nursing care field and others, systems for data measurement targeting human have been increasingly used. Such a system provides a user with valuable information for the user by calculating an analysis result from resultant data and feeding back the result to the user. The data is cyclic time-series data often.

As one example of such a system, a system (finger-tapping measuring/analyzing system) simply evaluating a cognitive function and a movement function by measuring and analyzing a finger-tapping motion of the user is exemplified (see, for example, a Patent Document 1).

In this specification, the finger-tapping motion means a repeatedly-opening/closing motion of a thumb and a forefinger. By the measurement of the finger-tapping motion, the cyclic time-series data is provided. It is known that success of the finger-tapping motion depends on presence/absence of and a severity of brain dysfunction such as dementia and Parkinson's disease. It has been pointed out that the analysis result of the cyclic time-series data measured by the system has possibilities of early recognition of such a brain dysfunction of the user, evaluation for estimation of the severity and others.

### RELATED ART DOCUMENT

### Patent Document

Patent Document 1: Japanese Patent Application Laid-Open Publication No. 2013-109540

### SUMMARY OF THE INVENTION

### Problems to be Solved by the Invention

It has been pointed out that the analysis result of the cyclic time-series data measured by the system has possibilities of early recognition of such a brain dysfunction of the user, evaluation for estimation of the severity and others. (in the present invention, this is referred to as (A) overall data evaluation that means evaluation for entire data waveform).

However, in a case of a bad evaluation result resulted from the analysis for the cyclic time-series data of the finger-tapping motion, a reason why this evaluation result is made could not have been offered so far. In other words, for the user, it could not have been explained which part of the cyclic time-series data is anomalous to cause the bad evaluation result, and therefore, conviction has not been made.

Therefore, a technique of detecting the anomaly part of the cyclic time-series data has been needed (in the present invention, the technique is referred to as (B) partial-data anomaly evaluation that means anomaly evaluation for a part of the data waveform).

However, a result of the (B) partial-data anomaly evaluation does not often match a result of the (A) overall-data evaluation. In other words, an evaluation model of the (A) is possibly inconsistent with an evaluation model of the (B) since the evaluation model of the (A) is created based on the cyclic time-series data itself while the evaluation model of the (B) is created based on the partial data extracted from the cyclic time-series data. The inconsistency between the (A) and the (B) makes the user confused of which one is reliable, and therefore, this inconsistency should be solved.

The inconsistency between the (A) and the (B) may be caused by the following two points of view. The first point of view is inconsistency in an anomaly rate. For example, circumstances on which the anomaly is detected in the (A) while the anomaly part is not detected in the (B) or opposite circumstances on which the anomaly is not detected in the (A) while a lot of anomaly parts are detected in the (B) are conceivable. Such inconsistent circumstances should not be caused. The second point of view is inconsistency in a contribution level of a feature amount to the anomaly detection. For example, circumstances on which a feature amount contributing to the anomaly detection in the (A) is not important in the anomaly detection in the (B) or opposite circumstances on which a feature amount not contributing to the anomaly detection in the (A) is important in the anomaly detection in the (B) are conceivable. Under such circumstances, an algorithm of the anomaly detection appears to be inconsistent, and there is a risk of losing the reliability of the entire system.

Accordingly, a purpose of the present invention is to provide highly-reliable evaluations of the overall data and the partial data in order not to cause the inconsistency in the above-described two points of view.

The above and other objects and novel characteristics of the present invention will be apparent from the description of the present specification and the accompanying drawings.

### Means for Solving the Problems

As means for solving the above-described issues, techniques described in the claims are used.

As one example cited, in a detecting apparatus detecting anomaly by using the cyclic information indicating a biological body state, the detecting apparatus includes: a cyclic-information acquiring unit configured to acquire the cyclic information; a cyclic-information feature-amount calculating unit configured to calculate a feature amount of the cyclic information acquired by the cyclic-information acquiring unit; a cyclic-information anomaly detecting unit configured to detect anomaly of the cyclic information based on the feature amount calculated by the cyclic-information feature-amount calculating unit; an anomaly-rate creating unit configured to create a cyclic-information anomaly rate based on a detection result of the cyclic-information anomaly detecting unit; a partial-information creating unit configured to create partial information based on the cycle by using the cyclic information acquired by the cyclic-information acquiring unit; a partial-information feature-amount calculating unit configured to calculate a feature amount of the partial information created by the partial-information creating unit; a partial-information anomaly detecting unit configured to detect anomaly of the partial information created by the partial-information creating unit, based on the feature amount calculated by the partial-information feature-amount calculating unit and the anomaly rate created by the anomaly-rate creating unit; and an output unit configured to output information based on a detection result of the partial-information anomaly detecting unit and a detection result of the cyclic-information anomaly detecting unit.

### Effects of the Invention

By usage of the techniques of the present invention, highly-reliable evaluations of overall data and partial data can be provided.

### BRIEF DESCRIPTIONS OF THE DRAWINGS

FIG. 1 is a configurational diagram of a human-data measuring system including a cyclic time-series data anomaly-part detecting system of a first embodiment;

FIG. 2 is a configurational diagram of the cyclic time-series data anomaly-part detecting system of the first embodiment;

FIG. 3 is a configurational diagram of a measuring apparatus of the first embodiment;

FIG. 4 is a configurational diagram of a terminal apparatus of the first embodiment;

FIG. 5 is a diagram showing a state in which a magnetic sensor that is a motion sensor is worn on a hand finger of a user;

FIG. 6 is a diagram showing a detailed configuration example of a motion-sensor control unit of a measuring apparatus or others;

FIG. 7 is a flowchart showing a procedure of entire process of the human-data measuring system of the first embodiment;

FIG. 8 is a diagram showing an example of a waveform signal of a feature amount;

FIG. 9 is a diagram showing an overall-data feature amount list;

FIG. 10 is a diagram continuously showing the overall-data feature amount list;

FIG. 11 is a diagram showing a feature-amount correspondence table;

FIG. 12 is a diagram continuously showing the feature-amount correspondence table;

FIG. 13 is a diagram showing a definition example of partial data;

FIG. 14 is a diagram showing a partial-data feature amount list;

FIG. 15 is a diagram showing an example of partial data detected as being anomaly;

FIG. 16 is a diagram for explaining an anomaly level;

FIG. 17 is a diagram for explaining a feature-amount contribution level;

FIG. 18 is a diagram showing a practice-menu list;

FIG. 19 is a diagram showing a practice-menu correspondence table;

FIG. 20 is a diagram showing an example of a menu screen that is an initial screen of a service;

FIG. 21 is a diagram showing a task measurement screen;

FIG. 22 is a diagram showing an evaluation result screen;

FIG. 23 is a diagram showing an anomaly-part detection result screen;

FIG. 24 is a diagram showing a cyclic time-series data anomaly-part detecting system of a second embodiment;

FIG. 25 is a diagram showing a configuration of a server; and

FIG. 26 is a diagram showing a data configurational example of user information managed in a DB by a server.

### BEST MODE FOR CARRYING OUT THE INVENTION

In the present working examples, a technique of detecting the anomaly part of the cyclic time-series data is proposed. Hereinafter, examples of embodiments of the present invention will be explained with reference to the accompanying drawings. Note that the same components are denoted by the same reference symbols in principle throughout all the drawings for explaining the embodiments, and the repetitive explanation thereof will be omitted.

The embodiments will be explained with reference to the drawings. However, the present invention is not interpreted to be limited to the contents described in the following embodiments. It could be easily understood for those who are skilled in the art that the specific configuration of the present invention is changeable with the scope of the idea or concept of the present invention.

If there are a plurality of elements having the same or similar function, the elements are denoted with the same symbol but the different index for the explanation. However, if it is unnecessary to discriminate the plurality of elements, the index is omitted for the explanation in some cases.

Terms such as “first”, “second”, and “third” in the present specification are attached in order to discriminate the elements, and are not always limit the numbers, the orders or the contents. The numbers for the identification of the elements are used for each phrase, and the number used in one phrase does not always indicate the same structure in a different phrase. Alternatively, the element identified by one number is not prevented from also having a function of the element identified by a different number.

A position, a size, a range and others of each component illustrated in the drawings or others are not often illustrated as a practical position, size, range and others in order to easily understand the invention. Therefore, the present invention is not always limited in the position, the size, the range and others disclosed in the drawings or others.

### First Embodiment

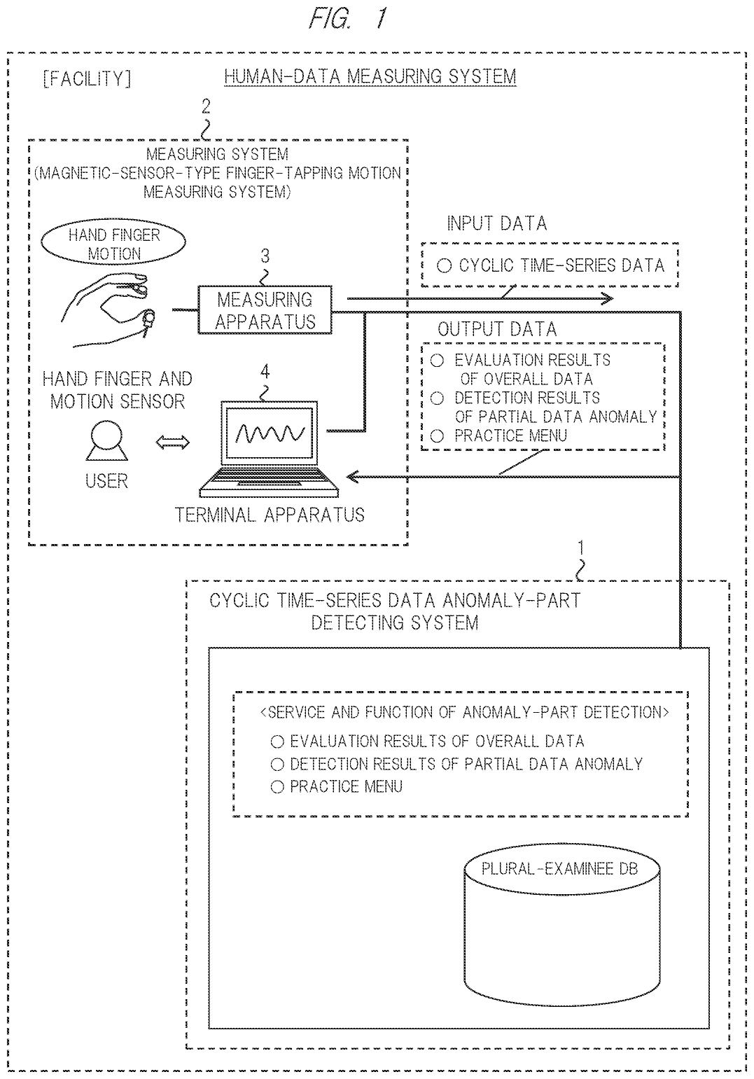

With reference to FIGS. 1 to 20, a cyclic time-series data anomaly-part detecting system (detecting apparatus) of a first embodiment will be explained. The cyclic time-series data anomaly-part detecting system of the first embodiment has a function of detecting an anomaly part of cyclic time-series data (that is cyclic information indicating a biological body state) resulted from measurement of an examinee. By this function, a detection result of the anomaly part of the cyclic time-series data can be matched with an evaluation result of the overall cyclic time-series data.

[Human-Data Measuring System]

FIG. 1 shows a configuration of a human-data measuring system including the cyclic time-series data anomaly-part detecting system of the first embodiment. In the first embodiment, the human-data measuring system is placed in a facility such as a hospital or a nursing care facility, a user's house or others. The human-data measuring system includes a cyclic time-series data anomaly-part detecting system 1 and a measuring system 2 that is a magnetic-sensor type finger-tapping motion system, and these systems are connected to each other through a communication line. The measuring system includes a measuring apparatus 3 and a terminal apparatus 4, and these apparatuses are connected to each other through a communication line. A plurality of measuring systems 2 may be placed in such a facility.

The measuring system 2 is a system of measuring a hand finger motion by using a magnetic-sensor type motion sensor. A motion sensor is connected to the measuring apparatus 3. This motion sensor is worn on the hand finger of the user. The measuring apparatus 3 measures the hand finger motion by using the motion sensor, and provides measuring data including a time-series waveform signal. The terminal apparatus 4 displays various types of information including the partial-data anomaly detection result on a display screen, and receives an operational input from the user. In the first embodiment, the terminal apparatus 4 is a PC.

The cyclic time-series data anomaly-part detecting system 1 has a function of offering an anomaly-part detecting service as a service based on information processing. Functions of the cyclic time-series data anomaly-part detecting system 1 include a partial-data anomaly detecting function. The partial-data anomaly detecting function is a function of detecting an anomaly part of the cyclic time-series data measured by the measuring system 2.

To the cyclic time-series data anomaly-part detecting system 1, for example, the cyclic time-series data or others is input as input data from the measuring system 2. From the cyclic time-series data anomaly-part detecting system 1, for example, the partial-data anomaly detection result or others is output as output data to the measuring system 2. The partial-data anomaly detection result includes a partial-data anomaly level and a partial-data anomaly feature amount in addition to the partial-data anomaly detection result.

The human-data measuring system of the first embodiment is applicable to not only the facility such as the hospital and the nursing care facility, the examinee and others, but also a wide variety of general facilities and people. The measuring apparatus 3 and the terminal apparatus 4 may be configured as a combined measuring system. The measuring system 2 and the cyclic time-series data anomaly-part detecting system 1 may be configured as a combined apparatus. The measuring apparatus 3 and the cyclic time-series data anomaly-part detecting system 1 may be configured as a combined apparatus.

[Cyclic Time-Series Data Anomaly-Part Detecting System]

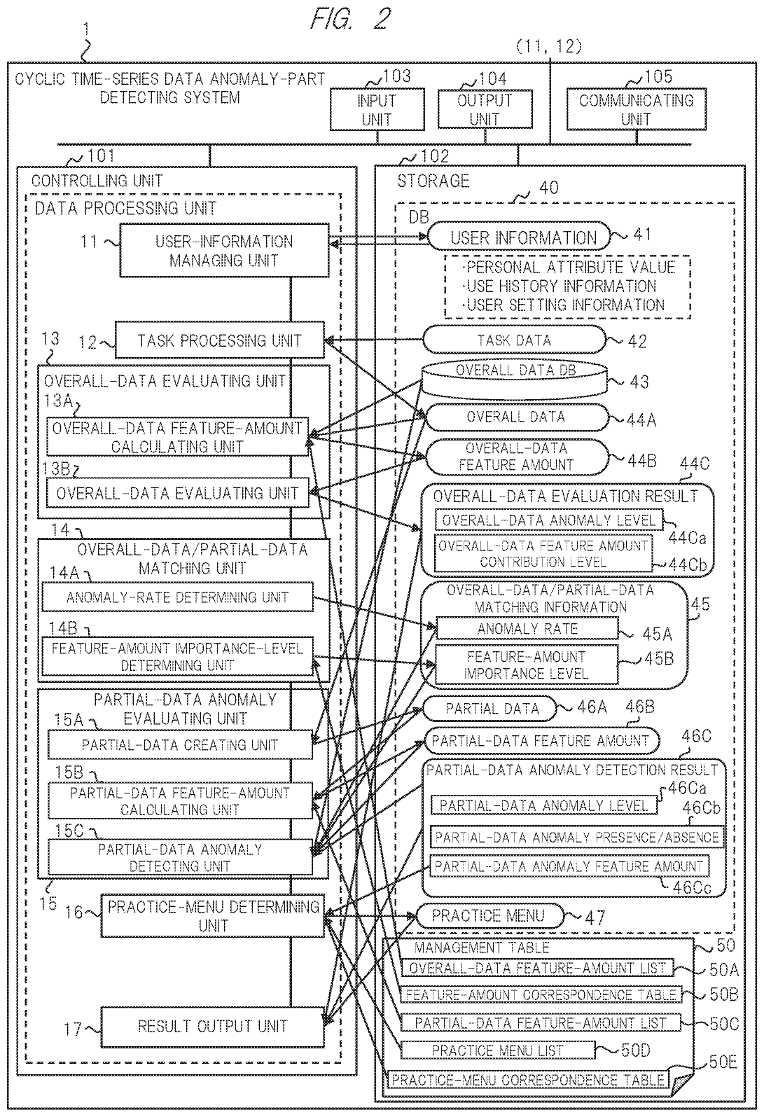

FIG. 2 shows a configuration of the cyclic time-series data anomaly-part detecting system 1 of the first embodiment. The cyclic time-series data anomaly-part detecting system 1 includes a control unit 101, a storage unit 102, an input unit 103, an output unit 104, a communication unit 105 and others, and these components are connected to each other through a bus. The input unit 103 is a unit on which the operational input is performed by an administrator of the cyclic time-series data anomaly-part detecting system 1 or others. The output unit 104 is a unit on which screen display or others is performed to the administrator of the cyclic time-series data anomaly-part detecting system 1 or others. The communication unit 105 is a unit including a communication interface performing a communication processing between the measuring apparatus 3 and the terminal apparatus 4.

The control unit 101 entirely controls the cyclic time-series data anomaly-part detecting system 1, is made of a Central Processing Unit (CPU), a Read Only Memory (ROM), a Random Access Memory (RAM) and others, and achieves a data processing unit performing the partial-data anomaly detection and others, based on a software program processing. The data processing unit of the control unit 101 includes a user-information managing unit 11, a task processing unit 12, an overall-data evaluating unit 13, an overall-data/partial-data matching unit 14, a partial-data anomaly evaluating unit 15, a practice-menu determining unit 16 and a result output unit 17. The control unit 101 achieves a function of inputting the measurement data from the measuring apparatus 3, a function of processing and analyzing the measurement data, a function of outputting a control instruction to the measuring apparatus 3 or the terminal apparatus 4, a function of outputting the display data to the terminal apparatus 4, and others.

The user-information managing unit 11 performs a processing of registering and managing the user information that is input by the user to user information 41 of a DB 40, a processing of checking the user information 41 of the DB 40 when the user uses the service, and others. The user information 41 includes a user's individual attribution value, use history information, user setting information and others. The attribution value includes a sex, an age and others. The use history information is information for managing a user's use history in the service offered by the present system. The user setting information is setting information for the functions of the present service set by the user.

The task processing unit 12 is a unit of performing a processing of a task for analyzing and evaluating a motility function and others. The task is, in other words, a predetermined hand finger motion. The task processing unit 12 outputs the task to the screen of the terminal apparatus 4 in accordance with task data 42 of the DB 40. The task processing unit 12 acquires the measurement data (the cyclic information indicating the biological body state) of the task measured by the measuring apparatus 3, and stores the data as the overall data 43A into the DB 40. The overall data described here means the overall cyclic time-series data measured during predetermined time. As described above, the task processing unit 12 (cyclic-information acquiring unit) acquires the cyclic information indicating the biological body state.

The overall-data evaluating unit 13 includes an overall-data feature-amount calculating unit 13A (that is a cyclic-information feature-amount calculating unit) and an overall-data evaluating unit 13B (that is a cyclic-information anomaly detecting unit). The overall-data feature-amount calculating unit 13A calculates a feature amount representing characteristics of overall data 44A (cyclic time-series data) in accordance with the overall data 44A of the user, and stores the feature amount as an overall-data feature amount 44B into the DB 40. The overall-data evaluating unit 13B evaluates the overall data in accordance with the overall-data feature amount 44B with reference to an overall-data DB 43, and stores an evaluation result as an overall-data evaluation result 44C into the DB 40. The overall-data evaluation result 44C is made of an overall-data anomaly level 44Ca and an overall-data feature-amount contribution level 44Cb.

The overall-data/partial-data matching unit 14 is made of an anomaly-rate determining unit 14A and a feature-amount importance-level determining unit 14B. The anomaly-rate determining unit 14A creates an anomaly rate 45A (cyclic-information anomaly rate) by using the overall-data anomaly level 44Ca, and stores the anomaly-rate data into the DB 40. The feature-amount importance-level determining unit 14B creates a feature-amount importance level 45B (feature-amount importance level) by using the overall-data feature-amount contribution level 44Cb with reference to a feature-amount correspondence table 50B, and stores the importance-level data into the DB 40. Combination of the anomaly rate 45A and the feature-amount importance level 45B is overall-data/partial-data matching information 45.

The partial-data anomaly evaluating unit 15 includes a partial-data creating unit 15A (that is a partial-information creating unit), a partial-data feature-amount calculating unit 15B (that is a partial-information feature-amount calculating unit), and a partial-data anomaly detecting unit 15C (that is a partial-information anomaly detecting unit). The partial-data creating unit 15A creates partial data 46A by dividing the overall data 44A, and stores the partial data into the DB 40. The partial-data feature-amount calculating unit 15B calculates a feature amount of each of the partial data 46A, and stores the feature amount data as a partial-data feature amount 46B into the DB 40. The partial-data anomaly detecting unit 15C determines the anomaly of the partial data in accordance with the partial-data feature amount 46B with reference to the partial data acquired from the overall-data DB 43 by using the anomaly rate 45A and the feature-amount importance level 45B, and stores the anomaly data as a partial-data anomaly detection result 46C into the DB 40. The partial-data anomaly detection result 46C includes a partial-data anomaly level 46Ca, a partial-data anomaly presence/absence 46Cb, and a partial-data anomaly feature amount 46Cc.

In this manner, the partial-data anomaly detecting unit 14C creates an anomaly level of the partial information created by the partial-data creating unit 15A, information indicating whether the partial information created by the partial-data creating unit 15A is anomalous or not, and information indicating an anomaly feature amount that is a feature amount to be a cause of the detection indicating that the partial information created by the partial-data creating unit 15A is anomalous. In this case, the cyclic time-series data anomaly-part detecting system 1 creates the detailed information of the anomaly of the partial information, and therefore, can further provide the detailed information by using the information allowing the part having the anomaly to be specified.

The practice-menu determining unit 16 determines a practice menu 47 based on a practice-menu list 50D and a practice-menu correspondence table 50E by using the partial-data anomaly feature amount 46Cc, and stores the practice menu data into the DB 40. In this manner, the practice-menu determining unit 16 determines the practice menu for improving the anomaly feature amount calculated by the partial-data anomaly detecting unit 14C.

The result output unit 17 performs a processing of outputting the overall-data evaluation result 44C, the partial-data anomaly detection result 46C and the practice menu 47 onto the screen of the terminal apparatus 4. The overall-data evaluating unit 13 and the partial-data anomaly evaluating unit 15 perform a screen output processing in coordination with the practice-menu determining unit 16 and the result output unit 17. In this manner, the result output unit 17 further outputs the menu determined by the practice-menu determining unit 16. In this case, the cyclic time-series data anomaly-part detecting system 1 provides the practice menu regarding the anomaly part of the partial data, and therefore, can provide the useful information for solving this anomaly part.

The result output unit 17 outputs the overall-data evaluation result 44C, and also outputs a result as a whole, and therefore, can provide information having a plurality of view points based on the cyclic time-series data.

The data and the information stored in the DB 40 of the storage unit 102 includes the user information 41, the task data 42, the overall-data DB 43, the overall data 44A, the overall-data feature amount 44B, the overall-data evaluation result 44C, the overall-data/partial-data matching information 45, the partial data 46A, the partial-data feature amount 46B, the partial-data anomaly detection result 46C, the practice menu 47 and others. By the control unit 101, a management table 50 is stored and managed in the storage unit 102.

The administrator can set contents of the management table 50. The management table 50 includes an overall-data feature-amount list 50A for setting the feature amount of the overall data, a feature-amount correspondence table 50B for setting correspondence between the feature amount of the overall data and the feature amount of the partial data, a partial-data feature-amount list 50C for setting the feature amount of the partial data, a practice-menu list 50D for setting a candidate of the practice menu, a practice-menu correspondence table 50E for setting correspondence between the partial-data anomaly feature amount 46Cc and the practice menu and others.

[Measuring Apparatus]

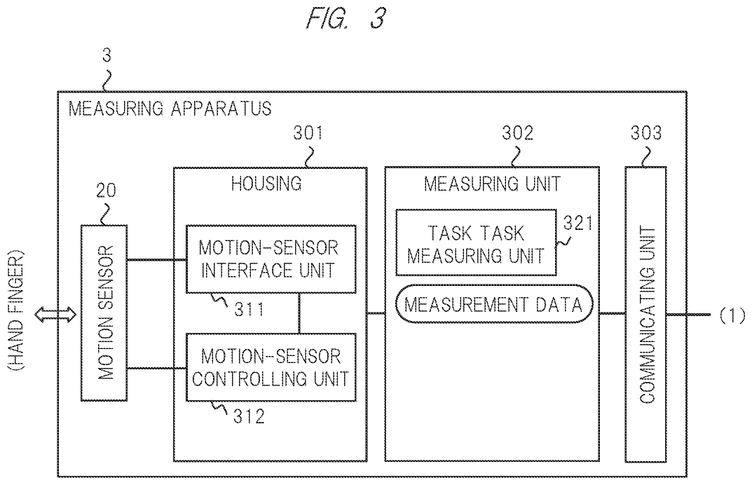

FIG. 3 shows a configuration of the measuring apparatus 3 of the first embodiment. The measuring apparatus 3 includes a motion sensor 20, a housing 301, a measuring unit 302, a communication unit 303 and others. The housing 301 includes a motion-sensor interface unit 311 connected to the motion sensor 20, and a motion-sensor control unit 312 controlling the motion sensor 20. The measuring unit 302 measures the waveform signal by using the motion sensor 20 and the housing 301, and outputs the waveform signal data as the measurement data. The measuring unit 302 includes a task measuring unit 321 for acquiring the measurement data. The communication unit 303 includes a communication interface, and communicates with the anomaly-data processing system 1 and transmits the measurement data to the anomaly-data processing system 1. The motion-sensor interface unit 311 includes an analog-digital converting circuit, and converts an analog waveform signal detected by the motion sensor into a digital waveform signal by sampling. The digital waveform signal is input to the motion-sensor control unit 312.

Note that a mode of storing each measurement data in storage means of the measuring apparatus 3 may be applicable, or a mode of storing each measurement data in not the measuring apparatus 3 but only the cyclic time-series data anomaly-part detecting system 1 may be applicable.

[Terminal Apparatus]

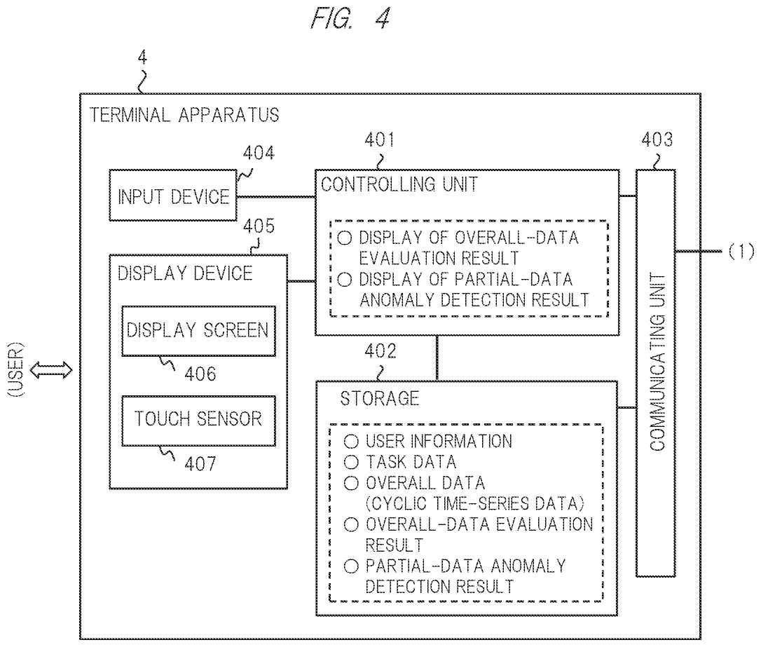

FIG. 4 shows a configuration of the terminal apparatus 4 of the first embodiment. The terminal apparatus 4 includes a control unit 401, a storage unit 402, a communication unit 403, an input device 404 and a display device 405. The control unit 401 performs display of the overall-data evaluation result, display of the partial-data anomaly detection result and others as a control processing based on a software program processing. The storage unit 402 stores the user information, the task data, the overall data (cyclic time-series data), the overall-data evaluation result, the partial-data anomaly detection result and others acquired from the cyclic time-series data anomaly-part detecting system 1. The communication unit 403 includes a communication interface, communicates with the cyclic time-series data anomaly-part detecting system 1, receives various data from the cyclic time-series data anomaly-part detecting system 1, and transmits a user's instruction input information or others to the cyclic time-series data anomaly-part detecting system 1. As the input device 404, a keyboard, a mouse and others are exemplified. In the display device 405, various information is displayed on a display screen 406. Note that the display device 405 may be a touch panel.

[Hand Finger, Motion Sensor, Finger-Tapping Measurement]

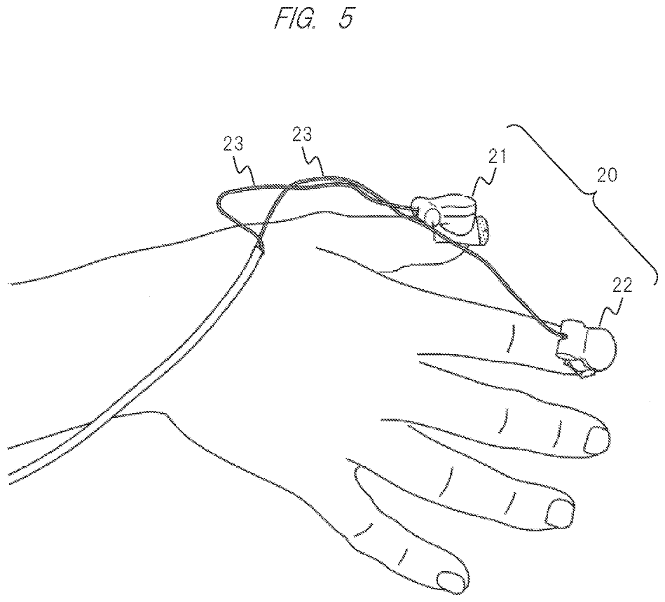

FIG. 5 shows a state in which a magnetic sensor that is the motion sensor 20 is worn on the hand finger of the user. The motion sensor 20 includes a transmitter coil unit 21 and a receiver coil unit 22 that are paired coils through a signal line 23 connected to the measuring apparatus 3. The transmitter coil unit 21 generates a magnetic field, and the receiver coil unit 22 detects this magnetic field. In an example of FIG. 5, the transmitter coil unit 21 is worn on a vicinity of a nail of a thumb of the user's right hand, and the receiver coil unit 22 is worn on a vicinity of a nail of a forefinger of the same. The wearing finger is changeable to a different finger. The wearing part is not limited to the vicinities of the nails, and any part is applicable.

As shown in FIG. 5, the motion sensor 20 is mounted to a target hand finger of the user, such as two fingers that are a thumb and a forefinger of a left hand. In this state, the user performs finger tapping that is a repeat motion of opening and closing the two fingers. As the finger tapping, a motion in change between a state of the closing two fingers that is a contact state of the two finger's tips and a state of the opening two fingers that is a separate state of the two finger's tips is performed. By the motion, an inter-coil distance between the transmitter coil unit 21 and the receiver coil unit 22, corresponding to a distance between the two finger's tips, is changed. The measuring apparatus 3 measures a waveform signal corresponding to the change of the magnetic field between the transmitter coil unit 21 and the receiver coil unit 22 of the motion sensor 20.

As the motion sensor 20, a different sensor from the magnetic sensor is applicable if the sensor can measure the distance between the two fingers. For example, the waveform of the distance between the two fingers can be provided by repeat opening and closing of the two fingers touching a tablet terminal or a touch panel type PC. Alternatively, the waveform of the distance between the two fingers can be provided by an infrared sensor detecting a shape of the hand or a position of the finger tip.

The finger-tapping motion specifically includes the following various tasks. As the motions, for example, one-hand free running motion, one-hand metronome motion, both-hand simultaneous free running motion, both-hand alternate free running motion, both-hand simultaneous metronome motion, both-hand alternate metronome motion, and others are exemplified. The one-hand free running motion means that two fingers of one hand perform the finger tapping many times as quick as possible. The one-hand metronome motion means that two fingers of one hand perform the finger tapping in synchronization with stimulation at a constant pace. The both-hand simultaneous free running motion means that two fingers of a left hand and two fingers of a right hand perform the finger tapping at the same timing. The both-hand alternate free running motion means that two fingers of a left hand and two fingers of a right hand perform the finger tapping at an alternate timing. Another motion is finger tapping following a marker.

[Motion-Sensor control Unit and Finger-Tapping Measurement]

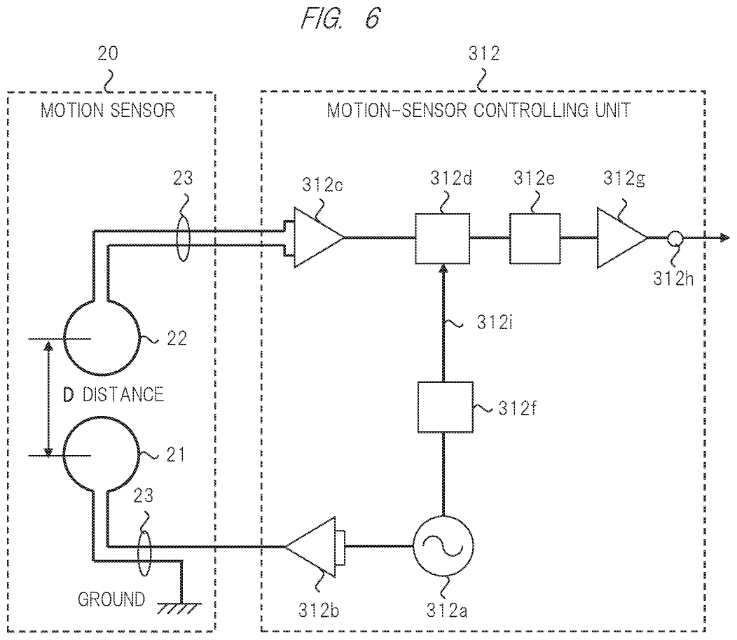

FIG. 6 shows a detailed configuration example of a motion-sensor control unit 312 of the measuring apparatus 3 or others. A distance “D” between the transmitter coil unit 21 and the receiver coil unit 22 in the motion sensor 20 is shown. The motion-sensor control unit 312 includes an alternating-current generator circuit 312a, a current-generating amplifier circuit 312b, a preamplifier circuit 312c, a wave-detector circuit 312d, an LPF circuit 312e, a phase adjuster circuit 312f, an amplifier circuit 312g, and an output-signal terminal 312h. To the generating amplifier circuit 312b and the phase adjuster circuit 312f are connected. To the current-generating amplifier circuit 312b, the transmitter coil unit 21 is connected through the signal line 23. To the preamplifier circuit 312c, the receiver coil unit 22 is connected through the signal line 23. At a post stage of the preamplifier circuit 312c, the wave-detector circuit 312d, the LPF circuit 312e, the amplifier circuit 312g, and the output-signal terminal 312h are sequentially connected. The wave-detector circuit 312d is connected to the phase adjuster circuit 312f.

The alternating-current generator circuit 312a creates an alternating-current voltage signal having a predetermined frequency. The current-generating amplifier circuit 312b converts the alternating-current voltage signal into an alternating current having a predetermined frequency, and outputs the alternating current to the transmitter coil unit 21. In the transmitter coil unit 21, the magnetic field is generated by the alternating current. This magnetic field generates the induced electromotive force in the receiver coil unit 22. The receiver coil unit 22 outputs an alternating current generated by the induced electromotive force. This alternating current has the same frequency as the predetermined frequency of the alternating-current voltage signal generated by the alternating-current generator circuit 312a.

The preamplifier circuit 312c amplifies the detected alternating current. The wave-detector circuit 312d performs the wave detection of the amplified signal in accordance with a reference signal 312i out of the phase adjuster circuit 312f. The phase adjuster circuit 312f adjusts a phase of the alternating-current voltage signal having the predetermined frequency or a double frequency out of the alternating-current generator circuit 312a, and outputs the signal as the reference signal 312i. The LPF circuit 312e limits a bandwidth of the wave-detected signal and outputs a resultant signal, and the amplifier circuit 312g amplifies this signal to have a predetermined voltage. Then, The output signal terminal 312h outputs an output signal corresponding to the measured waveform signal.

The waveform signal that is the output signal is a signal having a voltage value representing the distance D between the two fingers. The distance D and the voltage value are exchangeable in accordance with a predetermined calculus equation. This calculus equation can be also provided by calibration. The calibration is measured while, for example, the user holds a block having a predetermined length with two fingers of a target hand. The predetermined calculus equation is provided as an approximate curve that minimizes the error from a dataset of the voltage value and the distance value in the measurement value. Alternatively, by the calibration, a size of the user's hand may be recognized and used for normalization of the feature amount or others. In the first embodiment, the magnetic sensor is used as the motion sensor 20, and the measuring means handling the magnetic sensor is used. The present invention is not limited to this, and different detecting means and measuring means such as an acceleration sensor, a strain gauge and a high-speed camera are also applicable.

[Process Flow]

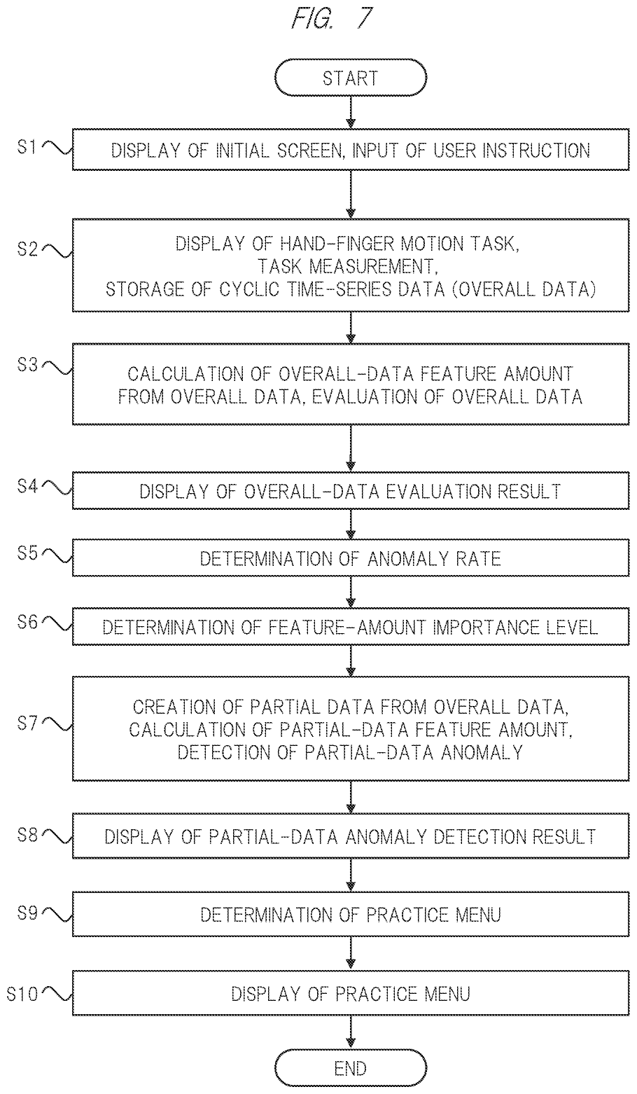

FIG. 7 shows a flow of entire process mainly performed by the cyclic time-series data anomaly-part detecting system 1 in the human-data measuring system of the first embodiment. The flow of FIG. 7 includes steps S1 to S10. The steps will be sequentially explained below.

(Step S1)

First, the user operates the measuring system 2. Specifically, in the terminal apparatus 4, the initial screen is displayed on the display screen. On the initial screen, the user selects a desirable operational item. For example, an operational item for detecting/processing the anomaly data is selected. The terminal apparatus 4 transmits the instruction input information corresponding to this selection to the cyclic time-series data anomaly-part detecting system 1. Alternatively, the user can enter and register the user information such as the sex, the age or others. In this case, the termina apparatus 4 transmits the entered user information to the cyclic time-series data anomaly-part detecting system 1. The user-information managing unit 11 of the cyclic time-series data anomaly-part detecting system 1 registers the user information into the user information 41.

(Step S2)

The task processing unit 12 of the cyclic time-series data anomaly-part detecting system 1 transmits the task data for the user to the terminal apparatus 4 in accordance with the instruction input information of the step S1 and the task data 42 of the finger tapping. The task data includes one or more types of the task information regarding the hand finger motion such as the one-hand free running motion, the both-hand simultaneous free running motion and the both-hand alternate free running motion. In the terminal apparatus 4, the task information of the hand finger motion is displayed on the display screen in accordance with the received task data. The user performs the task of the hand finger motion in accordance with the task information on the display screen. The measuring apparatus 3 measures the task, and transmits the measurement data to the cyclic time-series data anomaly-part detecting system 1. The cyclic time-series data anomaly-part detecting system 1 stores the measurement data into the measurement data 42B.

(Step S3)

The overall-data feature-amount calculating unit 13A of the cyclic time-series data anomaly-part detecting system 1 calculates the overall-data feature amount 44B by using the overall data 44A in accordance with the overall-data feature-amount list 50A. Then, the overall-data evaluating unit 13B provides the overall-data evaluation result 44C by applying a statistical method such as multivariate analysis, machine learning or others to the overall-data feature amount 44B. The overall-data evaluation result 44C includes the overall-data anomaly level 44Ca and the overall-data feature-amount contribution level 44Cb.

(Step S4)

The result output unit 17 of the cyclic time-series data anomaly-part detecting system 1 transmits the overall-data evaluation result 44C to the terminal apparatus 4, and this result is displayed on the screen. In this manner, the result output unit 17 outputs the information based on the detection result made by the overall-data evaluating unit 13B. On the screen, the user can check the evaluation result of the user's cyclic time-series data.

(Step S5)

The anomaly-rate determining unit 14A of the cyclic time-series data anomaly-part detecting system 1 calculates the anomaly rate 45A in accordance with the overall-data anomaly level 44Ca.

(Step S6)

The anomaly-rate determining unit 14A of the cyclic time-series data anomaly-part detecting system 1 calculates the feature-amount importance level 45B in accordance with the overall-data feature-amount contribution level 44Cb with reference to the feature-amount correspondence table 50B.

(Step S7)

The partial-data creating unit 15A of the cyclic time-series data anomaly-part detecting system 1 creates the partial data 46A by using the overall data 44A. The partial-data creating unit 15A creates the partial information based on the cycle (such as information of one cycle) by using the overall data 44A. Then, the partial-data feature-amount calculating unit 15B calculates the partial-data feature amount 46B by using the partial data 46A in accordance with the partial-data feature-amount list 50C. Then, the partial-data anomaly detecting unit 15C provides the partial-data anomaly detection result 46C by applying the statistical method such as the multivariate analysis or the machine learning to the partial-data feature amount 46B while using the anomaly rate 45A and the feature-amount importance level 45B.

(Step S8)

The result output unit 17 of the cyclic time-series data anomaly-part detecting system 1 transmits the partial-data anomaly detection result 46C to the terminal apparatus 4, and the result data is displayed on the screen. On the screen, the user can check the anomaly part of the user's cyclic time-series data.

(Step S9)

The practice-menu determining unit 16 of the cyclic time-series data anomaly-part detecting system 1 creates the practice menu 47 in accordance with the partial-data anomaly feature amount 46Cc with reference to the practice-menu list 50D and the practice-menu correspondence table 50E.

(Step S10)

The result output unit 17 of the cyclic time-series data anomaly-part detecting system 1 transmits the practice menu 47 to the terminal apparatus 4, and the menu data is displayed on the screen. On the screen, the user can check the practice menu to be performed by the user.

[Calculation of Overall-Data Feature Amount]

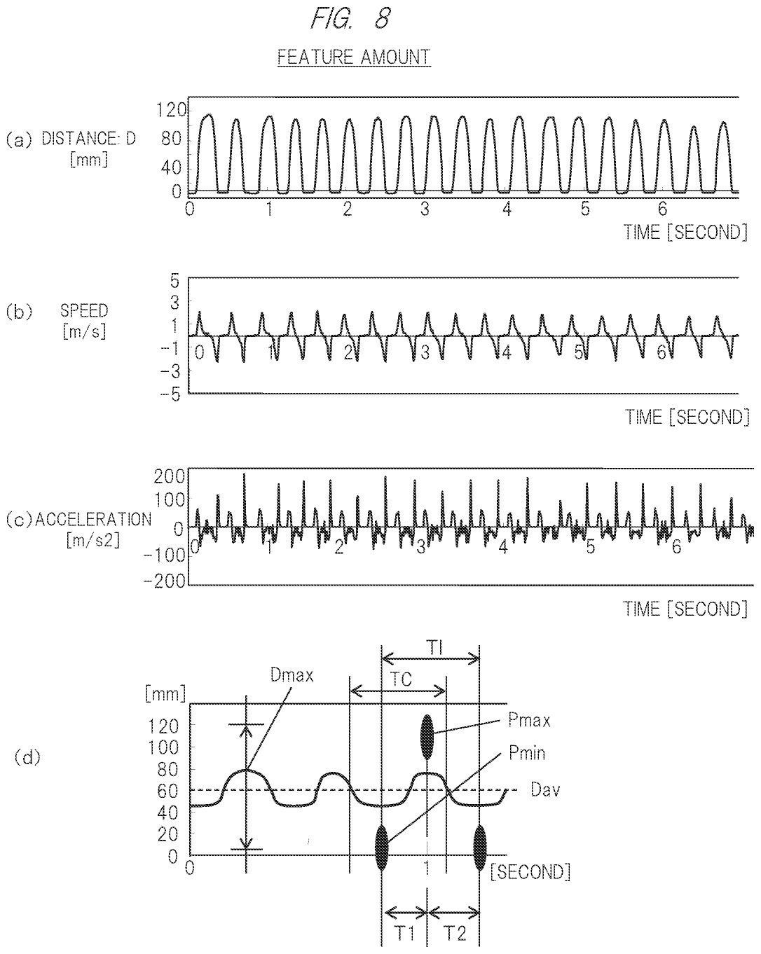

FIG. 8 shows an example of the waveform signal of the feature amount. FIG. 8(a) shows a waveform signal of the distance D between the two fingers, FIG. 8(b) shows a waveform signal of a speed of the two fingers, and FIG. 8(c) shows a waveform signal of an acceleration of the two fingers. The speed of FIG. 8(b) is provided by temporal differentiation of the waveform signal of the distance of FIG. 8(a). The acceleration of FIG. 8(c) is provided by temporal differentiation of the waveform signal of the speed of FIG. 8(b). The overall-data feature-amount calculating unit 13A provides a waveform signal of a predetermined feature amount as described in the present example, in accordance with the calculation such as differentiation, integration or others by using the waveform signal of the overall data 44A. The overall-data feature-amount calculating unit 13A provides a value based on the predetermined calculation by using the feature amount.

FIG. 8(d) shows an example of the feature amount in broad interpretation of FIG. 8(a). This shows the maximum value Dmax of the distance D of the finger tapping, a tap interval TI and others. A horizontal broken line indicates an average value Dav of the distance D in entire measurement time. The maximum value Dmax indicates the maximum value of the distance D in entire measurement time. The tap interval TI indicates time for a cycle TC of one finger tapping, and particularly indicates time from the local minimum point Pmin to the next local minimum point Pmin. In addition, this indicates the local maximum point Pmax and the local minimum point Pmin in one cycle of the distance D, and time T1 of the opening motion and time T2 of the closing motion described later.

The detailed example of the feature amount will be further described below. In the first embodiment, a plurality of feature amounts resulted from the waveforms of the distance, the speed and the acceleration are used. In another embodiments, note that only some feature amounts of the plurality of feature amounts may be used, or different feature amounts may be used, and the details of the definition of the feature amounts is not limited, either.

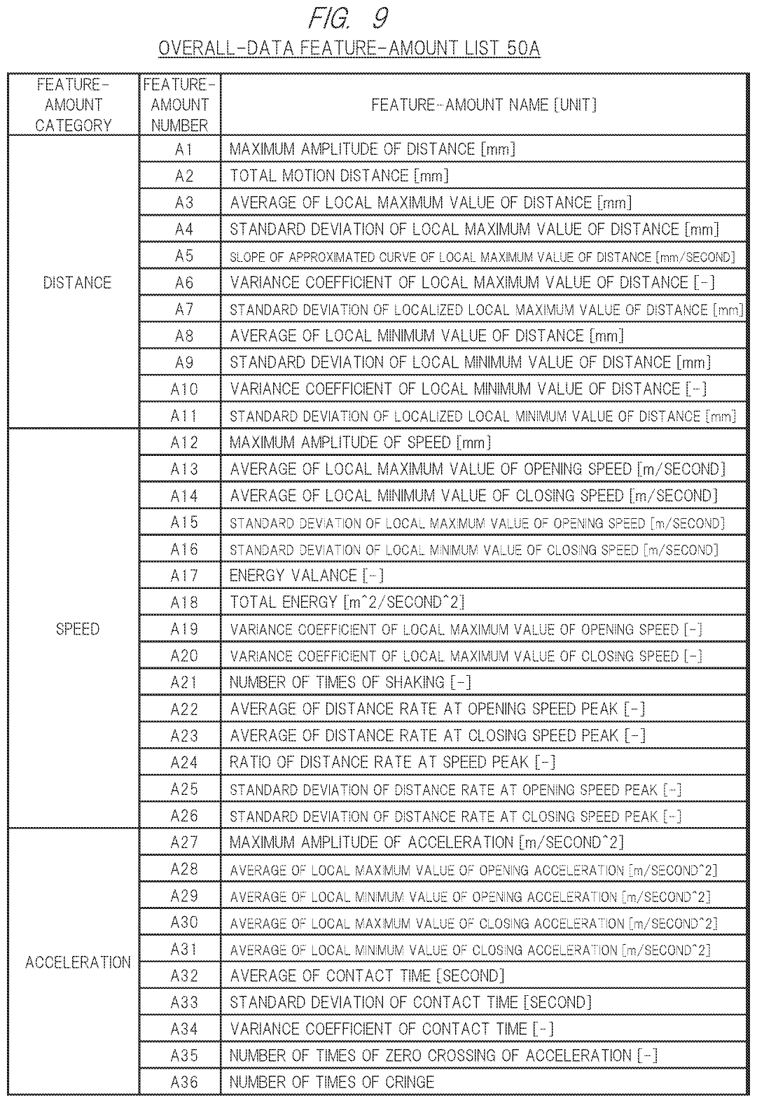

FIG. 9 is a diagram showing the overall-data feature-amount list 50A. The setting for the linking is one example, and is changeable. A row of the overall-data feature-amount list 50A of FIG. 9 includes a feature-amount category, an identification number and a feature-amount parameter. The feature-amount category includes [Distance], [Speed], [Acceleration], [Tap Interval], [Phase Difference], and [Marker Following].

For example, the feature amount [Distance] includes a plurality of feature-amount parameters identified with identification numbers (A1) to (A11). A term in parentheses [ ] of the feature-amount parameter indicates a unit. (A1) “Maximum Amplitude of Distance” [mm] is a difference between the maximum value and the minimum value of the amplitude of the waveform of the distance (FIG. 8(a)). (A2) “Total Motion Distance” [mm] is a sum of absolute values of distance change amounts in entire measurement time of one measurement.

(A3) “Average of Local Maximum Values of Distance” [mm] is an average of the local maximum values of the amplitudes of the respective cycles. (A4) “Standard Deviation of Local Maximum Values of Distance” [mm] is a standard deviation of the above-described values. (A5) “Slope (Damping Rate) of Approximate Curve of Local Maximum Points of Distance” [mm/second] is a slope of a curve of approximated local maximum points of the amplitudes. This parameter mainly represents amplitude change due to fatigue during the measurement time. (A6) “Variance Coefficient of Local Maximum Values of Distance” is a variance coefficient of the local maximum values of the amplitudes, and its unit ([−]) represents dimensionless quantity. This parameter is a value that is the normalized standard deviation by average, and therefore, personal difference in a finger length can be excluded. (A7) “Standard Deviation of Localized Local Maximal Values of Distance” [mm] is a standard deviation of local maximal values of three adjacent amplitudes.

This parameter is a parameter for evaluating a degree of amplitude variation in local short time. (A8) “Average of Local Minimum Values of Distance” [mm] is an average of the local minimum values of the amplitudes of the respective cycles. (A9) “Standard Deviation of Local Minimum Values of Distance” [mm] is a standard deviation of the above-described values. (A10) “Variance Coefficient of Local Minimum Values of Distance” is a variance coefficient of the local minimum values of the amplitudes, and its unit ([−]) represents dimensionless quantity. This parameter is a value that is the normalized standard deviation by average, and therefore, personal difference in a finger length can be excluded. (A11) “Standard Deviation of Localized Local Minimum Values of Distance” [mm] is a standard deviation of local minimum values of three adjacent amplitudes. This parameter is a parameter for evaluating a degree of localized short-time amplitude variation.

The feature amount [Speed] includes feature-amount parameters identified by the following identification numbers (A12) to (A26). (A12) “Maximum Amplitude of Speed” [m/second] is a difference between the maximum value and the minimum value of the speed in the waveform of the speed (FIG. 8(b)). (A13) “Average of Local Maximum Values of Opening Speed” [m/second] is an average of the local maximum values of the speed at the time of the opening motion in the respective finger tapping waveforms. The opening motion is a motion of the two fingers from the closing state to the maximum opening state (FIG. 8(d)). (A14) “Average of Local Minimum Values of Closing Speed” [m/second] is an average of the local minimum values of the speed at the time of the closing motion. The closing motion is a motion of the two fingers from the maximum opening state to the closing state. (A15) “Standard Deviation of Local Maximum Values of Opening Speed” [m/second] is a standard deviation of the maximum values of the speed at the time of the opening motion.

(A16) “Average of Local Minimum Values of Closing Speed” [m/second] is an average of local minimum values of the speed at the time of the closing motion. (A17) “Energy Balance” [−] is a ratio between a sum of squares of the speeds during the opening motion and a sum of squares of the speeds during the closing motion. (A18) “Total Energy” [m²/second²] is a sum of squares of the speed during the entire measurement time. (A19) “Variance Coefficient of Local Maximum Values of Opening Speed” [−] is a variance coefficient of the local maximum values of the speed at the time of the opening motion, and is a value that is normalized standard deviation by average. (A20) “Average of Local Minimum Values of Closing Speed” [m/second] is an average of local minimum values of the speeds at the time of the closing motion. (A21) “Number of Times of Shaking” [−] is a number provided by subtraction of the number of times of the finger tapping with the large opening/closing motion from the number of times of reciprocation changing in a sign of the waveform value of the speed between positive and negative. (A22) “Average of Distance Rate at Opening Speed Peak” [−] is an average value of rates of distances each having the maximum speed value during the opening motion provided when the amplitude of the finger tapping is set to 1.0. (A23) “Average of Distance Rate at Closing Speed Peak” [−] is an average value of the similar rates of distances each having the minimum speed value during the closing motion. (A24) “Ratio between Distance Rates at Speed Peak” [−] is a ratio between the value of (A22) and the value of (A23). (A25) “Standard Deviation of Distance Rate at Opening Speed Peak” [−] is a standard deviation of the rates of the distances each having the maximum speed value during the opening motion provided when the amplitude of the finger tapping is set to 1.0. (A26) “Standard Deviation of Distance Rate at Closing Speed Peak” [−] is a standard deviation of the similar rates of the distances each having the minimum speed value during the closing motion.

The feature amount [Acceleration] includes feature-amount parameters identified by the following identification numbers (A27) to (A36). (A27) “Maximum Amplitude of Acceleration” [m/second²] is a difference between the maximum value and the minimum value of the acceleration in the waveform of the acceleration (FIG. 8(c)). (A28) “Average of Local Maximum Values of Opening Acceleration” [m/second²] is an average of the local maximum values of the acceleration during the opening motion, and is a first value of four extremums. (A29) “Average of Local Minimum Values of Opening Acceleration” [m/second²] is an average of the local minimum values of the acceleration during the opening motion, and is a second value of the four extremums.

(A30) “Average of Local Maximum Values of Closing Acceleration” [m/second²] is an average of the local maximum values of the acceleration during the closing motion, and is a third value of the four extremums. (A31) “Average of Local Minimum Values of Closing Acceleration” [m/second²] is an average of the local minimum values of the acceleration during the closing motion, and is a fourth value of the four extremums. (A32) “Average of Contact Time” [second] is an average of contact time in the closing state of the two fingers. (A33) “Standard Deviation of Contact Time” [second] is a standard deviation of the contact time. (A34) “Variance Coefficient of Contact Time” [second] is a variance coefficient of the contact time. (A35) “Number of Times of Zero Crossing of Acceleration” [−] is an average number of times of sign change of the acceleration value between positive and negative during one cycle of the finger tapping. This value is ideally two. (A36) “Number of Times of Cringe” [−] is a value provided by subtraction of the number of times of the finger tapping with the large opening/closing from the number of times of the reciprocation changing in the sign of the acceleration value between positive and negative during one cycle of the finger tapping.

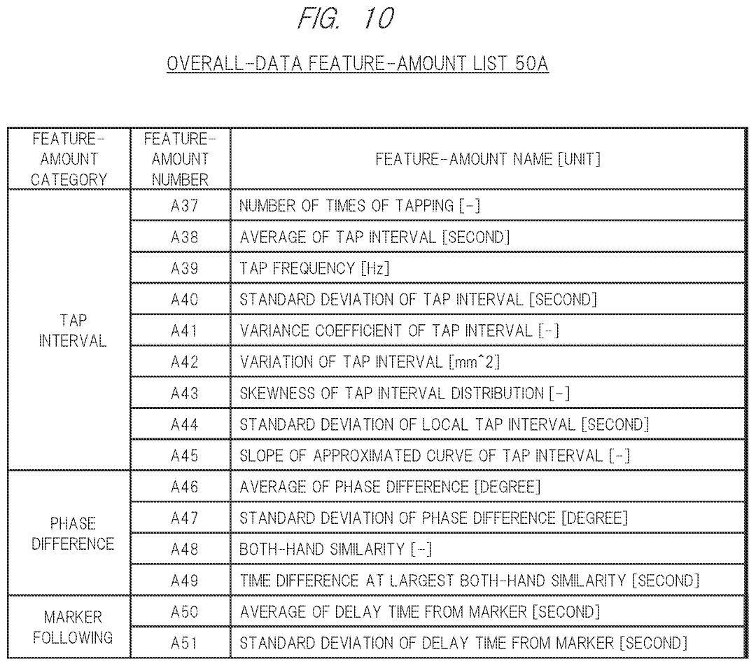

Next, FIG. 10 is a diagram continuously showing the overall-data feature-amount list 50A. The feature amount [Tap Interval] includes a plurality of feature-amount parameters identified by the following identification numbers (A37) to (A45). (A37) “Number of Times of Tapping” [−] is the number of times of the finger tapping during the entire measurement time of one measurement. (A38) “Tap-Interval Average” [second] is an average of the tap intervals (FIG. 8(d)) in the waveform of the distance. (A39) “Tap Frequency” [Hz] is a frequency having the maximum spectrum in Fourier transform of the waveform of the distance. (A40) “Tap-Interval Standard Deviation” [second] is a standard deviation of the tap intervals. (A41) “Tap-Interval Variance Coefficient” [−] is a variance coefficient of the tap intervals, and is a value that is the normalized standard deviation by average. (A42) “Variation of Tap Interval” [mm²] is a cumulative value in frequencies of 0.2 to 2.0 Hz in spectrum analysis of the tap interval.

(A43) “Skewness of Tap-Interval Distribution” [−] is a skewness of a frequent distribution of the tap interval, and represents a skewness level of the frequent distribution in comparison to normal distribution. (A44) “Standard Deviation of Local Tap Intervals” [second] is a standard deviation of three adjacent tap intervals. (A45) “Slope (Damping Rate) of Approximate Curve of Tap Intervals” [−] is a slope of a curve of an approximated tap interval. This slope mainly represents tap-interval change due to fatigue during the measurement time.

The feature amount [Phase Difference] includes a plurality of feature-amount parameters identified by the following identification numbers (A46) to (A49). (A46) “Average of Phase Difference” [degree] is an average of phase differences in the waveform of the both hands. The phase difference is an indicator for mismatch of the left-hand finger tapping with the right-hand finger tapping as shown with an angle when one cycle of the right-hand finger tapping is set to 360°. A state without the mismatch is set to 0°. (A47) “Standard Deviation of Phase Difference” [degree] is a standard deviation of the phase differences. The larger the value of (A46) or the value of (A47) is, the larger the mismatch between the both hands is, and the more the instability is. (A48) “Both-Hand Similarity” [−] is a value representing correlation provided when time mismatch is 0 in a case of application of a cross-correlation function to the right-hand and left-hand waveforms. (A49) “Time Mismatch at Maximum Both-Hand Similarity” [second] is a value representing time mismatch at the maximum correlation of (A48).

The feature amount [Marker Following] includes a plurality of feature-amount parameters identified by the following identification numbers (A50) and (A51). (A50) “Average of Delay Time from Marker” [second] is an average of delay time of the finger tapping from time at which a marker is cyclically shown. The marker is dealt with by stimuli such as visual stimulus, audio stimulus and tactual stimulus. This parameter value is based on a moment of the closing state of the two fingers. (A51) “Standard Deviation of Delay Time from Marker” [second] is a standard deviation of the delay time.

[Overall-Data Evaluation]

The overall-data evaluating unit 13B provides the overall-data evaluation result 44C representing good/bad of the overall data in accordance with the overall-data feature amount 44B calculated by the overall-data feature-amount calculating unit 13A. For example, with reference to the overall-data DB 43, an estimate equation for estimating the anomaly is provided by multiple regression analysis taking the anomaly as an objective variable and taking the plurality of feature amounts of the overall-data feature amount 44B as an explanatory variable. The anomaly is defined as an indicator that is small in the normal state while large in the anomalous state. As an example of the anomaly, a severity score of the brain dysfunction or others is exemplified, such as the Mini Mental State Examination (MMSE) representing a severity of the dementia and the Unified Parkinson's Disease Rating Scale (UPDRS) representing a severity of the Parkinson's disease. However, such a severity has characteristics in which the more the normal state is, the larger the value of the severity is, while the more the anomalous state is, the smaller the value is. For example, in the MMSE, a perfect score that is 30 points shows the highest cognitive function, and, the closer to zero the score is, the lower the cognitive function is. Therefore, the MMSE or the UPDRS is used for the anomaly after a preprocessing of inverting a positive sign and a negative sign of the MMSE or the UPDRS. Then, the overall-data feature amount 44B is assigned to the estimate equation of the multiple regression analysis, and an estimated severity score is provided as the overall-data anomaly level 44Ca.

The overall-data feature-amount contribution level 44Cb has a larger value when having larger influence on the estimation model of each feature amount, and has a smaller value when having a smaller influence of the same. For example, as the overall-data feature-amount contribution level 44Cb, an absolute value of a standardized partial regression coefficient of the estimate equation of the multiple regression analysis is designed.

In order to estimate the anomaly, not the multiple regression analysis but a similar method may be used. For example, a discriminant/regression method of simultaneously performing discrimination and regression based on a linear model may be used. Alternatively, a support vector machine regression method or a different regression method such as a neural network may be used.

The overall-data anomaly level 44Ca may be not the severity score of the brain dysfunction when being an indicator representing a mismatch level from the normal finger-tapping waveform. The overall-data feature-amount contribution level 44Cb may be not the standardized partial regression coefficient when being an indicator representing an importance level of each feature amount of the overall-data feature amount 44B in the estimate equation.

[Determination of Anomaly Rate]

The anomaly-rate determining unit 14A provides an anomaly rate 45A (R[%]) by assigning the overall-data anomaly level 44Ca (X) to a predetermined conversion function. The term “R” is designed to “0% R 100%”. The conversion function is a function that monotonically increases so that the larger the overall-data anomaly level 44Ca is, the larger the R is, and, for example, an exponent function of “R =a * exp(X−b) +c” is set. The term “a” is designed to be a large value when it is desirable to rapidly increase the R along with increase in the X, or a small value when it is desirable to monotonically increase the R along with the increase in the X. And, in this conversion function, the terms “b” and “c” are designed so as to satisfy “R=0%” when the anomaly level (after the preprocess) is the expectable minimum value (such as −30 in MMSE) or satisfy “R=Rm (0% Rm≤100%, such as Rm=50%)” when the anomaly level (after the preprocess) is the expectable maximum value (such as 0 in MMSE). In this manner, the partial-data anomaly detecting unit 15C does not detect the anomaly at all when the anomaly level is at the minimum, and detects the more anomalies when the anomaly level is larger.

Note that the overall-data evaluating unit 13B may estimate the overall-data anomaly level 44Ca out of the range of the expectable minimum to maximum values (−30 to 0 in the MMSE) of the anomaly level (after the preprocess). In this case, the overall-data anomaly level may be changed to the minimum value when being smaller than the minimum value or to the maximum value when being larger than the maximum value. Note that the conversion function may be not the exponent function if being the monotonically increase function, and may be, for example, a logarithm function, a sigmoid function, a linear function or others.

[Determination of Feature-Amount Importance Level]

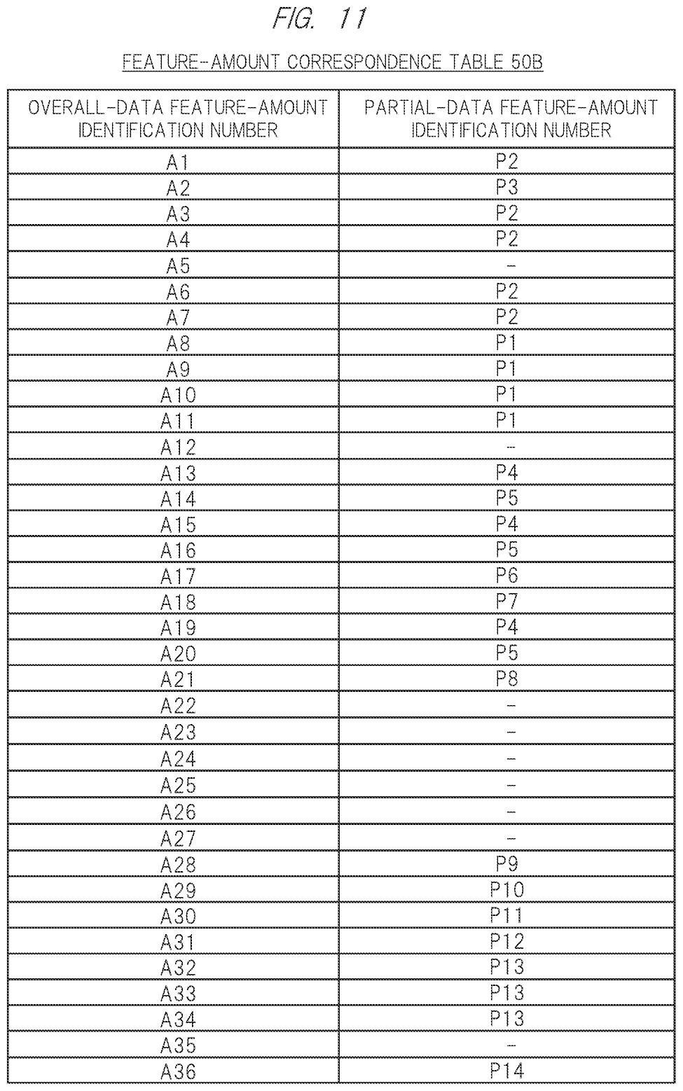

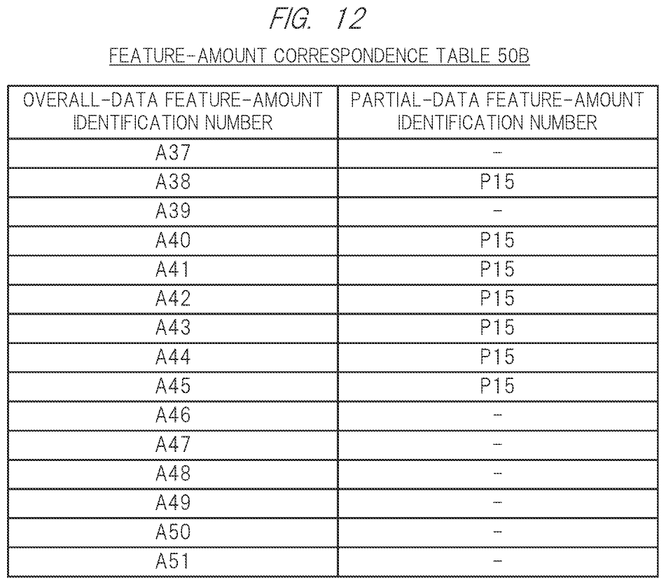

With reference to the feature-amount correspondence table 50B shown in FIGS. 11 and 12, the feature-amount importance-level determining unit 14B provides a feature-amount importance level (Qk (k=1, 2, . . . and NP (the number of the partial-data feature amounts)) 45B by using the overall-data feature-amount contribution level 44Cb. First, one overall-data feature amount Aj is selected from the overall-data feature-amount list 50A, and a corresponding partial-data feature amount Pk is searched with reference to the feature-amount correspondence table 50B. For example, (P2) “Maximum Value of Distance” corresponds to (A1) “Maximum Amplitude of Distance”.

Then, the feature-amount importance level Qk is provided by assigning the overall-data feature-amount contribution level Cj (j=1, 2, . . . and NA (the number of the overall-data feature amounts)) of the overall-data feature amount Aj to the predetermined conversion function. The conversion function is a function that monotonically increases so that the larger the overall-data feature-amount contribution level Cj is, the larger the feature-amount importance level Qk is, and is set to be, for example, an exponent function. This conversion function is designed to satisfy “Qk=1” when the Cj is the expectable minimum value or satisfy “Qk=100” that is a larger value when the Cj is the expectable maximum value. In this manner, the partial-data anomaly detecting unit 15C performs the anomaly detection without focusing the Pk when the overall-data feature-amount contribution level Cj is the minimum value, or performs the anomaly detection with more focusing the Pk when the overall-data feature-amount contribution level Cj is larger. Note that the conversion function may be not the exponent function if being the monotonically increase function, and may be, for example, a logarithm function, a sigmoid function, a linear function or others. When the above-described processes are performed to all the overall-data feature-amount contribution levels Cj, all the feature-amount importance levels Qk can be provided. Note that a plurality of Cj are corresponded to the same Qk in some cases. In this case, the Qk may be calculated after the largest Cj is selected. The present invention is not limited to this, the Qk may be calculated after the largest Cj is selected, or the Qk may be calculated from an average value of the plurality of Cj. Alternatively, when none of the Cj is corresponded to the Qk, “Qk=1” may be set as a default value.

[Creation of Partial Data]

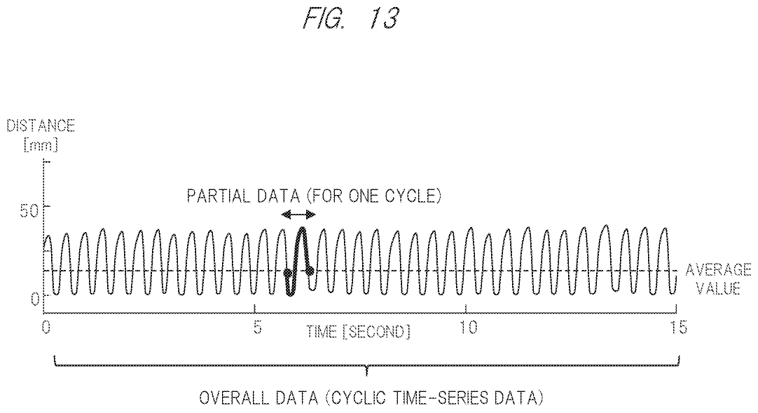

The partial-data creating unit 15A extracts the finger-tap waveform for each cycle to provide the partial data 46A. As shown in FIG. 13, in order to extract the partial data 46A, one cycle of the finger tapping is defined from a moment of downward crossing on the average of the overall data 44A to a next moment of downward crossing on the same. Since the one cyclic is defined with respect to the average of the overall data 44A as described above, uncompleted up and down motions that are not acceptable as the finger-tapping motion can be eliminated, the uncompleted up and down motions being a case of the too small distance value (local maximum value) in the end of the opening of the two fingers and a case of the too large distance value (local minimum value) in the end of the closing of the two fingers. The definition method of the one cyclic may be a different method, and it may be defined from a moment of the local minimum point to a moment of the next local minimum point or from a moment of the local maximum point to a moment of the next local maximum point. As the method of extracting the partial data 46A, the partial data 46A may be extracted for not each cycle but each of a plurality of cycles to be divided.

A later-described partial-data feature-amount calculating unit 15B also calculates the feature amounts (P19) and (P20) using the waveforms of the both hands. However, in order to calculate these feature amounts, the waveforms of the both hands in the same time zone are necessary. Therefore, one cyclic may be extracted in the right-hand waveform, and a waveform in the same time zone may be extracted from a left-hand waveform. This approach to the right hand and the left hand may be inverted.

[Calculation of Partial-Data Feature Amount]

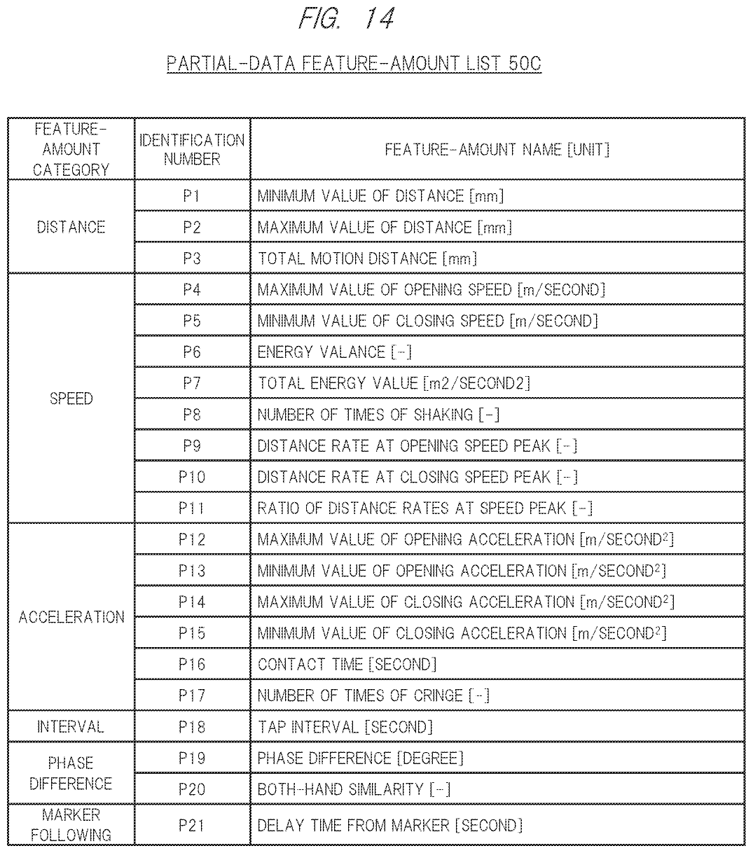

FIG. 14 shows the partial-data feature-amount list 50C. With reference to this list, the partial-data feature-amount calculating unit 15B calculates the partial-data feature amount 46B. Its row includes a feature-amount category, an identification number and a feature-amount parameter. The feature-amount category includes [Distance], [Speed], [Acceleration], [Tap Interval], [Phase Difference] and [Marker Following]. The partial-data feature-amount calculating unit 15B may calculate all feature amounts of the partial-data feature-amount list 50C or select some feature amounts to be calculated.

For example, the feature amount [Distance] includes a plurality of feature-amount parameters identified with identification numbers (P1) to (P3). A term in parentheses [ ] of the feature-amount parameter indicates a unit. (P1) “Minimum Value of Distance” [mm] is the minimum value of the amplitude of the partial data. (P2) “Maximum Value of Distance” [mm] is the maximum value of the amplitude of the partial data. (P3) “Total Motion Distance” [mm] is a sum of absolute values of distance change amounts during entire measurement time of the partial data.

The feature amount [Speed] includes feature-amount parameters identified with identification numbers (P4) to (P8). (P4) “Maximum Value of Opening Speed” [m/second] is the maximum value of the speed in the opening motion of the partial data. The opening motion is a motion from the closing state of the two fingers to the maximum opening state. (P5) “Minimum Value of Closing Speed” [m/second] is the minimum value of the speed in the closing motion. The closing motion is a motion from the maximum opening state of the two fingers to the closing state. (P6) “Energy Balance” [−] is a ratio between a sum of squares of the speed in the opening motion and a sum of squares of the speed in the closing motion. (P7) “Total Energy” [m²/second²] is a sum of squares of the speed during the entire measurement time of the partial data. (P8) “Number of Times of Shaking” [−] is a count provided by subtraction of 1 that is the number of times of the finger tapping from the number of times of reciprocation changing in a sign of the waveform value of the speed between positive and negative. (P9) “Distance Rate at Opening Speed Peak” [−] is a distance at the maximum speed value in the opening motion when the amplitude of the finger tapping is set to 1.

(P10) “Distance Rate at Closing Speed Peak” [−] is a distance at the minimum speed value in the closing motion when the amplitude of the finger tapping is set to 1. (P11) “Ratio between Distance Rates at Speed Peak” [−] is a ratio between the value of (P10) and the value of (P11).

The feature amount [Acceleration] includes feature-amount parameters identified with the following identification numbers (P12) to (P17). (P12) “Maximum Value of Opening Acceleration” [m/second²] is the maximum value of the acceleration in the opening motion, and is the first value of four extremums. (P13) “Minimum Value of Opening Acceleration” [m/second²] is the minimum value of the acceleration in the opening motion, and is the second value of the four extremums. (P14) “Maximum Value of Closing Acceleration” [m/second²] is the maximum value of the acceleration in the closing motion, and is the third value of the four extremums. (P15) “Minimum Value of Closing Acceleration” [m/second²] is the minimum value of the acceleration in the closing motion, and is the fourth value of the four extremums. (P16) “Contact Time” [second] is contact time of the two fingers in the closing state. (P17) “Number of Times of Cringe” [−] is a value provided by subtraction of 1 that is the number of times of the finger tapping with the large opening/closing from the number of times of the reciprocation changing in the sign of the acceleration value between positive and negative during one cycle of the finger tapping.

The feature amount [Tap Interval] includes a feature-amount parameter identified with the following identification number (P18). (P18) “Tap Interval” [second] is time for one cyclic of the finger tapping.

The feature amount [Phase Difference] includes feature-amount parameters identified with the following identification numbers (P19) to (P20). (P19) “Phase Difference” [degree] is a phase difference of the waveforms of the both hands. When one cyclic of the right-hand finger tapping is set to 360 degrees, the phase difference is an indicator representing a difference of the left-hand finger tapping from the right-hand one as an angle. In a case without the difference, the angle is set to 0 degree. (P20) “Both-Hand Similarity” [−] is a value representing correlation in a case without time difference of 0 under application of a cross-correlation function to the waveforms of the right hand and the left hand.

The feature amount [Marker Following] includes a feature-amount parameter identified with the following identification number (P21). This feature amount is calculated with reference to a motion task following a marker. (P21) “Delay Time from Marker” [second] is delay time of the finger tapping from time at which the marker is cyclically shown. The marker is dealt with by stimuli such as visual stimulus, audio stimulus and tactual stimulus. This parameter value is based on a moment of the closing state of the two fingers.

[Partial-Data Anomaly Detection]

The partial-data anomaly detecting unit 15C detects the anomaly of the partial data 46A by using the multivariate analysis or the machine learning. As a preprocess, first, the partial-data feature amount 46B is standardized so that the average is 0 while the standard deviation is 1 as generally performed. By such standardization, weights of the feature amounts in the model resulted from the multivariate analysis or the machine learning can be prevented from being not uniform due to the difference in the range of each feature amount. Next, a feature-amount spatial distribution of the partial-data feature amount 46B is changed with reference to the feature-amount importance level (Qk) 45B that is calculated by the feature-amount importance-level determining unit 14B. As one example of a method of changing the feature-amount spatial distribution, the standardized partial-data feature amount Pk is multiplied by the feature-amount importance level Qk (k=1, 2 . . . and NP (the number of the partial-data feature amounts)). By this process, the distribution of the partial-data feature amount having the high importance level is made large in the feature-amount space, and its anomaly due to the machine learning can be easily detected. Alternatively, as another example of the method of changing the feature-amount spatial distribution, the partial-data feature amount 46B (Ak) may be assigned to an exponent function “Ak'=p*Qk*exp(Ak)” (the term “p” is a predetermined value) to change the Ak into the Ak′. In this manner, the farther from the average 0 the partial-data feature amount 46B (Ak) is, the farther the far data is. The larger the feature-amount importance level Qk is, the rapidly farther the far data is, and therefore, the data can be easily detected as the anomaly.

Then, 1-class Support Vector Machine (SVM) that is one type of the machine learning is used to perform the anomaly detection. The SVM to be a precondition of this process is a method in 2-class classification of defining a classification boundary so as to maximize a margin between the classification boundary (a hyperplane expressed by a linear function) and data of each class. However, when the classification boundary being the hyper plane cannot be separated if the classification boundary of two groups has a complicate shape, and therefore, the SVM is devised to be capable of handling the complicate-shape classification boundary by application of a kernel function. An idea of a 1-class SVM is the same as that of the 2-class classification of the SVM, but is a method of classifying the data into the anomaly data at a certain rate and other normal data in one class. A rate of outliers of the 1-class SVM is designed to be the anomaly rate 45A (R) calculated by the anomaly-rate determining unit 14A. In this manner, the higher the overall-data anomaly level 44Ca is, the larger the rate of the partial data to be detected as the anomaly is.

Note that a different method from the 1-class SVM may be applied to the detection of the anomaly of the partial data. For example, a normal distribution centering the average of the feature-amount distribution of the partial data 46A may be assumed, and data having a large distance from the center of the normal distribution may be detected as the anomaly.

[Partial-Data Anomaly Detection Result]

In the 1-class SVM, a classification score “y” is calculated, and data having a classification score y of a negative value is determined as the anomaly. A result detected by this determination is designed to be the partial-data anomaly presence/absence 46Cb. It is conceivable that the farther from 0 and the smaller the classification score y is, the larger the anomaly level is. Accordingly, this classification score y is transformed by a function of causing “z=0%” in “y=0” and “z=100%” in “y =−∞” to be asymptotic, and the term “z” is designed to be the partial-data anomaly level 46Ca. And, in order to find out which feature amount of all feature amounts contributes to the anomaly determination of the finger-tapping waveform for each cycle determined as the anomaly by the 1-class SVM, a feature amount that is far from the average by the standard deviation “SD=2.0 or more” is designed to be the partial-data anomaly feature amount 46Cc.

[Effect of Partial-Data Anomaly Evaluating Unit]

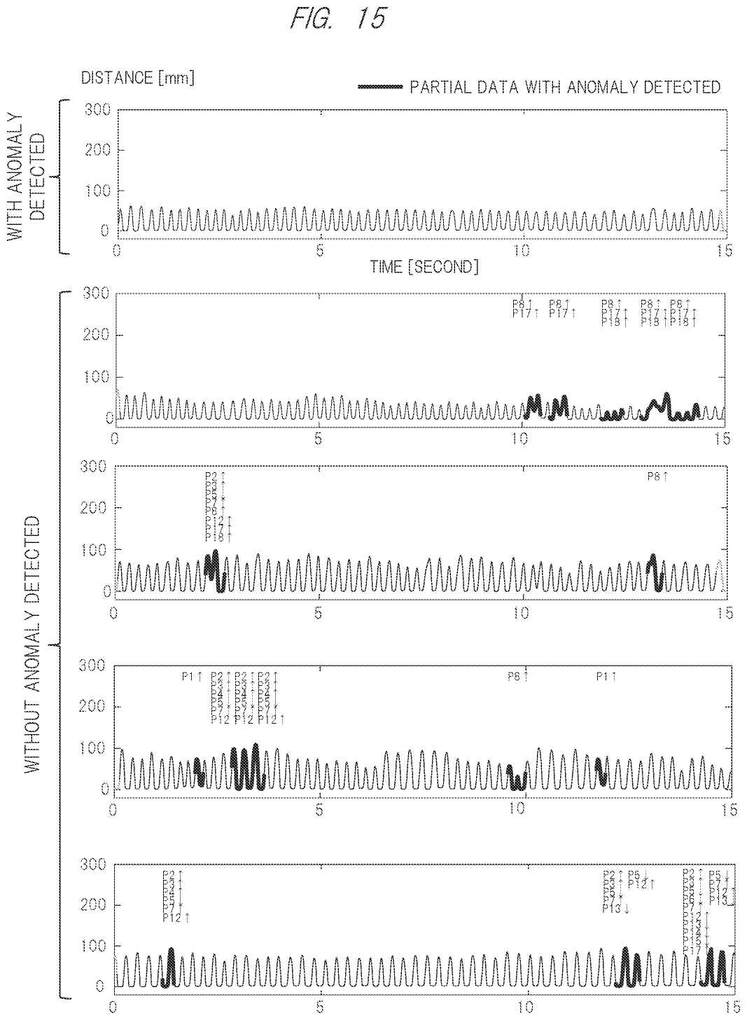

An example of the partial-data anomaly detection result 46C is shown in FIG. 15. On the waveform of the distance of the finger-tapping motion, the partial data 46A that is determined as the anomaly by the partial-data anomaly presence/absence 46Cb is shown with a thick line and overlapped. Above this, the partial-data anomaly feature amount 46Cc is shown. The top graph shows the overall data 44A with none of the anomaly-detected partial data 46A. The lower four graphs show the overall data 44A with one anomaly-detected partial data 46A or more.

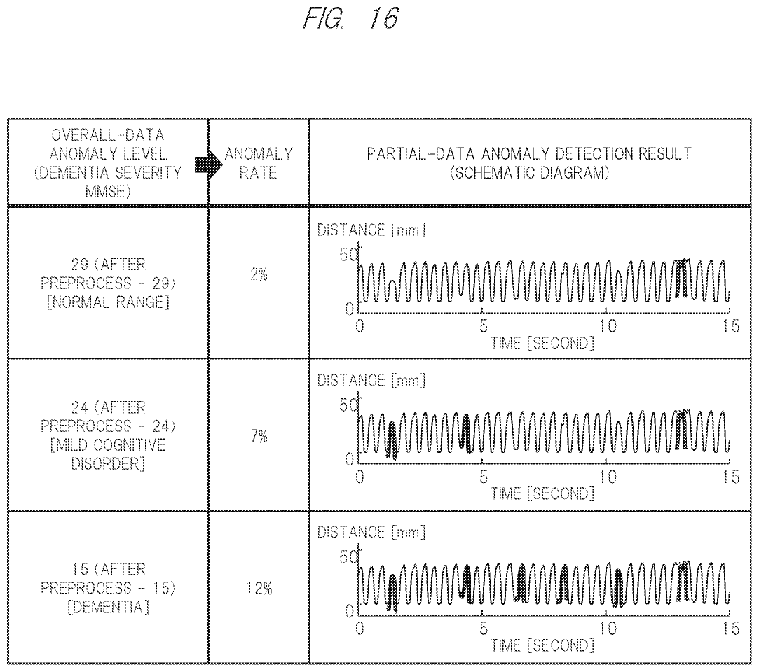

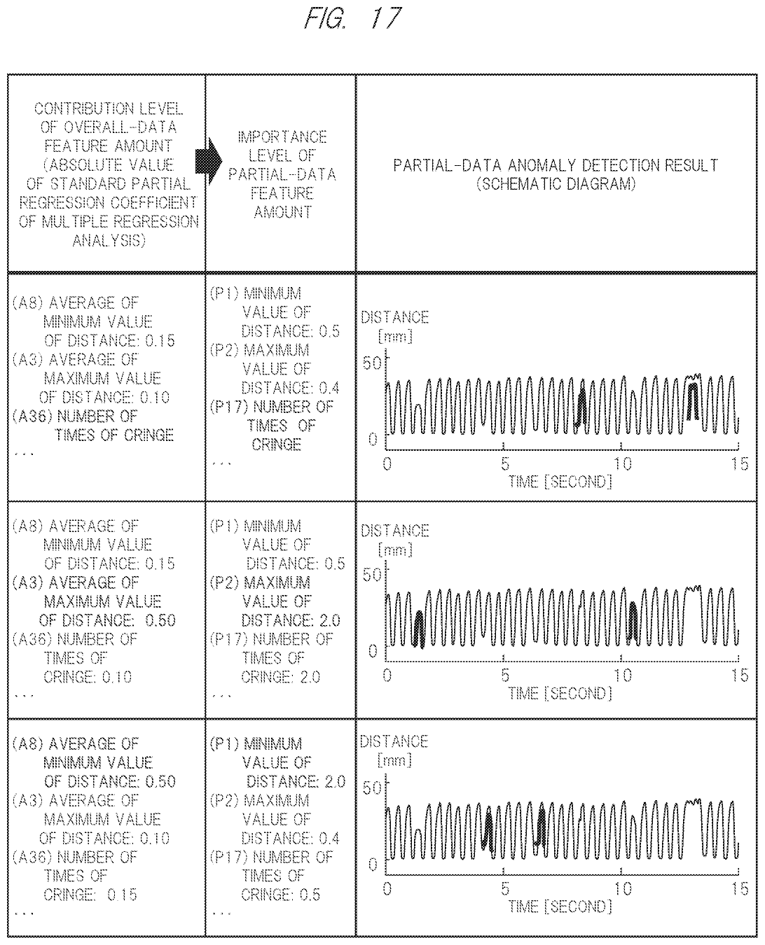

Each of FIGS. 16 and 17 is a schematic view understandably showing the effects caused by the application of the anomaly-rate determining unit 14A and the feature-amount importance-level determining unit 14B.

FIG. 16 shows samples of the partial-data anomaly detection result 46C in a case of the anomaly rate 45A having a different value. For example, when the overall-data anomaly level 44Ca (the dementia severity MMSE) is 29, the anomaly rate is determined to be 2% by the anomaly-rate determining unit 14A. In other words, the partial-data anomaly detecting unit 15C detects 2% of all cycles during the measurement time as the anomaly, and detects only one piece of the partial data of the waveforms. Next, when the overall-data ano1maly level 44Ca is 24, the anomaly rate is calculated to be higher (7%) than that of the case of “29”, and three pieces of the partial data are detected as the anomaly in the waveforms. At last, when the overall-data anomaly level 44Ca is 15, the anomaly rate is calculated to be further higher (12%) than the above-described cases, and six pieces of the partial data are detected as the anomaly in the waveforms. As described above, in the anomaly detection of the partial data, the corresponding anomaly rate to the anomaly detection result of the overall data can be set by the application of the anomaly-rate determining unit 14A.

FIG. 17 shows the partial-data anomaly detection result 46C in a case of the feature-amount importance level 45B having a different value. In this example, for easily understanding this case, only three feature amounts of the overall-data feature-amount list 50A are selected to show the overall-data feature-amount contribution level 44Cb. In the uppermost example, the overall-data feature-amount contribution level 44Cb of (A36) “Number of Times of Cringe” is 0.50, and is higher than those of other feature amounts. This value is applied to the feature-amount importance-level determining unit 14B, so that the feature-amount importance level 45B of the partial data is provided. As a result, the feature-amount importance level 45B of (P17) “Number of Times of Cringe” is the largest, and therefore, the anomaly detection is performed to focus on the partial data having the anomaly value in (P17) “Number of Times of Cringe”. Next, in the second example, the overall-data feature-amount contribution level 44Cb of (A3) “Average of Maximum Value of Distance” is 0.50, and is higher than those of other feature amounts. Accordingly, the feature-amount importance level 45B of (P2) “Maximum Value of Distance” is the largest, and therefore, the anomaly detection is performed to focus on the partial data having the anomaly value in (P2) “Maximum Value of Distance”. At last, in the third example, the overall-data feature-amount contribution level 44Cb of (A8) “Average of Minimum Value of Distance” is 0.50, and is higher than those of other feature amounts. Accordingly, the feature-amount importance level 45B of (P1) “Minimum Value of Distance” is the largest, and therefore, the anomaly detection is performed to focus on the partial data having the anomaly value in (P1) “Minimum Value of Distance”. As described above, by the application of the feature-amount importance-level determining unit 14B, the feature amount contributing to the anomaly determination of the overall data and the feature amount of the corresponding partial data are weighed, so that the anomaly detection can be performed to focus on the anomaly partial data having the same characteristics as those of the anomaly of the overall data.

As described above, by the anomaly-rate determining unit 14A and the feature-amount importance-level determining unit 14B, the anomaly detection can be performed on the partial data (the finger-tapping waveform for each cycle) while the matching with the anomaly detection result of the overall data is maintained.

[Determination of Practice Menu]

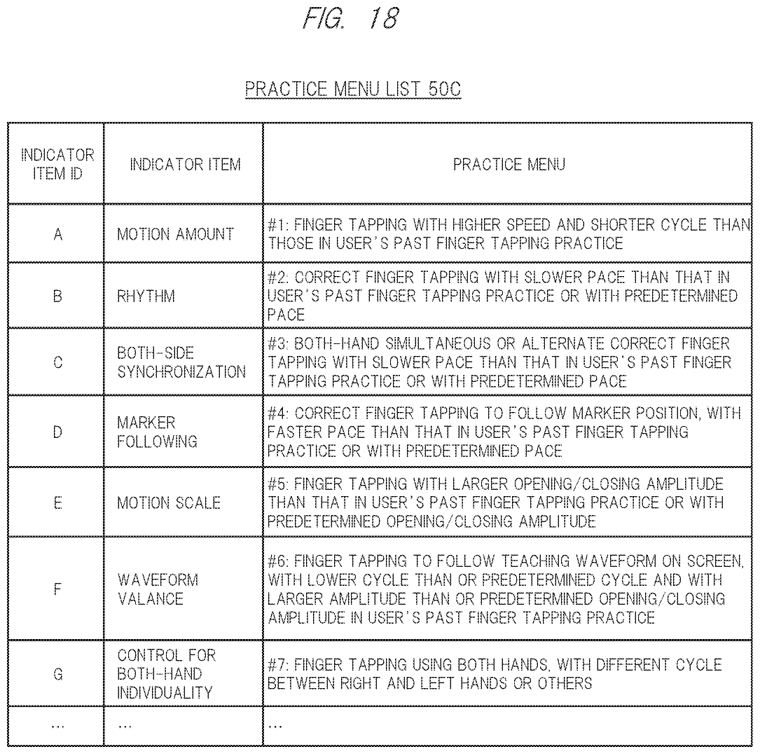

FIG. 18 shows the practice menu list 50C showing the indicator items representing the characteristics of the finger-tapping motion and the practice menu for improving the indicator items. The indicator items include [Motion Amount], [Endurance], [Rhythm], [Both-Side Synchronization], [Marker Following], [Motion Scale], [Waveform Balance] and [Amplitude Control]. The setting for the indicator items and the practice menu is one example, and is changeable.

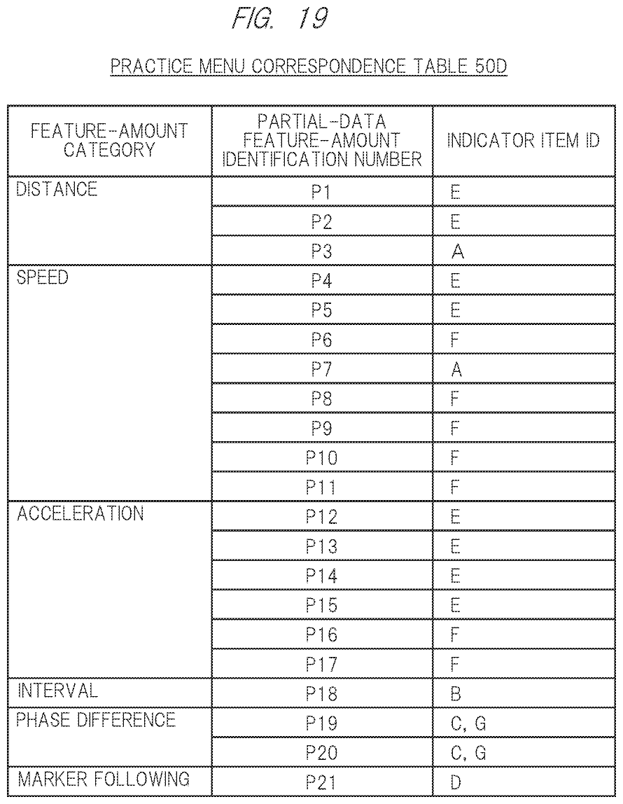

FIG. 19 shows the practice-menu correspondence table 50D related to the information for setting the correspondence between the feature amount and the practice menu item. This setting for the correspondence is one example, and is changeable. A row of this table includes the feature-amount category, the identification number, the feature-amount parameter and the indicator item. The feature-amount category includes [Distance], [Speed], [Acceleration], [Tap Interval], [Phase Difference] and [Marker Following]. The feature amount of this list matches that of the partial-data feature-amount list 50C, and is corresponded to at least one or more of the indicator items that are set in the practice menu list 50D.

[Display Screen (1)—Menu]

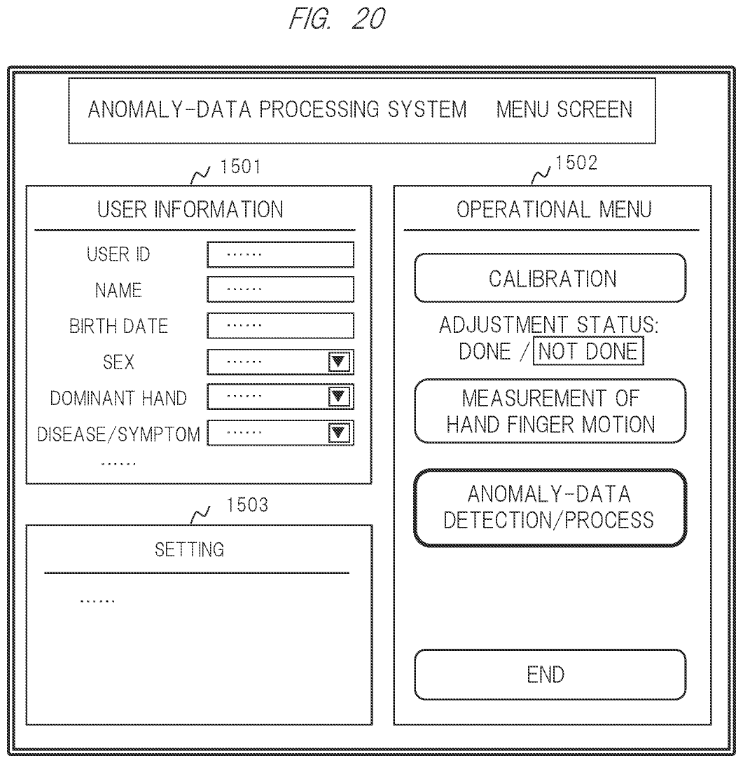

As an example of a display screen of the terminal apparatus 4, FIG. 20 shows an example of a menu screen that is an initial screen of the service. This menu screen includes a user information section 1501, an operational menu section 1502, a setting section 1503 and others.

In the user information section 1501, the user information is input by the user and is registerable. If the user information is already input to an electronic health record or others, the section may be in corporation with this user information. As examples of the enterable user information, a user ID, a name, a birth date or an age, a sex, a dominant hand, disease/symptom, a note and others are exemplified. The dominant hand is selectable from and enterable as a right hand, a left hand, both hands, uncertain and others. The disease/symptom may be selectable from and enterable as, for example, an option in a list box, or may be enterable as an optional text. When this system is used in a hospital or others, not the user but a doctor or others may enter them instead of the user. The present anomaly-data processing system is also applicable to a case without the registration of the user information.

In the operational menu section 1502, operational items for functions offered as the service are displayed. The operational items include “Calibration”, “Measurement of Hand Finger Motion”, “Anomaly-Data Detection/Process”, “End” and others. When the “Calibration” is selected, the above-described calibration, in other words, the process for the adjustment of the motion sensor 20 or others to the user's hand finger is performed. A status indicating whether the adjustment is done or not is also displayed. When the “Measurement of Hand Finger Motion” is selected, the screen changes to a task measurement screen for measuring the task of the hand finger motion such as the finger tapping. When the “Anomaly-Data Detection/Process” is selected, the anomaly detection is performed to the measured data to be targeted, the anomaly-data detection result is displayed, and the screen changes to a screen for performing the process to the detected anomaly data. When the “End” is selected, the service ends.

In the setting section 1503, the user setting is enabled. For example, when there is a type of the anomaly detection item to be detected by the user, the measuring operator or the administrator, the anomaly detection item can be selected from the options and can be set. And, a process for each anomaly detection item can be selected. A threshold of the anomaly data detection or others can be also set. These setting contents are transmitted to the cyclic time-series data anomaly-part detecting system 1 through the communicating unit 105, and the cyclic time-series data anomaly-part detecting system 1 detects/processes the anomaly data with reference to the specified setting in this stage.

[Display Screen (2)—Task Measurement]

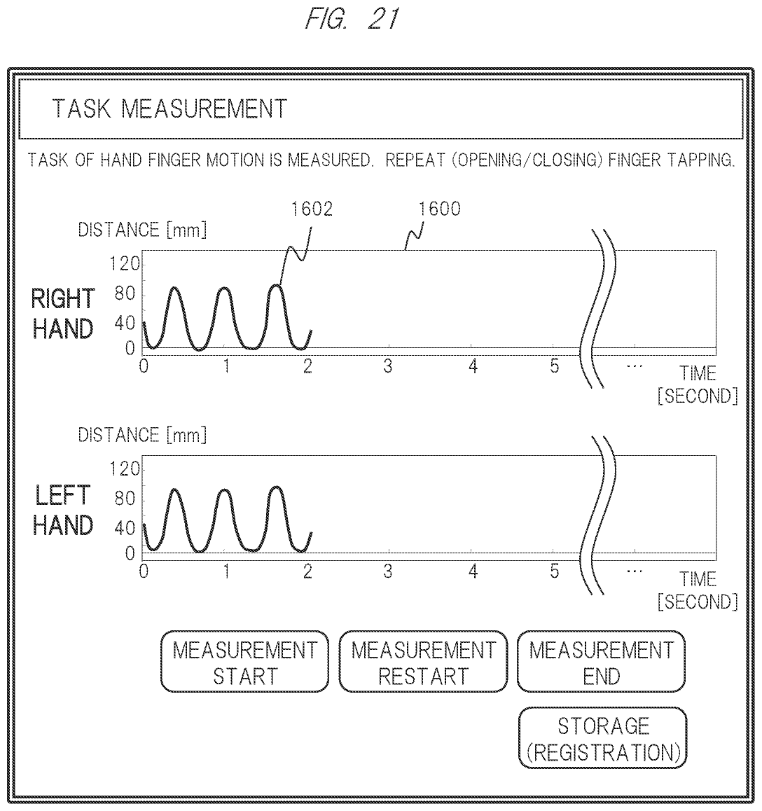

As another example, FIG. 21 shows a task measurement screen. This screen displays task information. For example, regarding each of the right and the left hands, this displays a graph 1600 taking the time on a horizontal axis and the distance between the two fingers on a vertical axis. On the screen, another teaching information for explaining the task content may be output. For example, a video region for explaining the task content by using image/audio may be arranged. Inside the screen, operational buttons for “Measurement Start”, “Measurement Restart”, “Measurement End”, “Storage (Registration)” and others are arranged, and the user can select the buttons. The user selects the “Measurement Start” in accordance with the task information on the screen, and performs the motion of the task. The measuring apparatus 3 measures the motion of the task, and provides the waveform signal. On the graph 1600, the terminal apparatus 4 displays a measured waveform 1602 corresponding to the waveform signal in the measurement in real time. The user selects the “Measurement End” after the motion, and selects the “Storage (Registration)” when determining the data. The measuring apparatus 3 transmits the measured data to the anomaly-data processing system 1.

[Display Screen (3)—Overall-Data Evaluation Result]

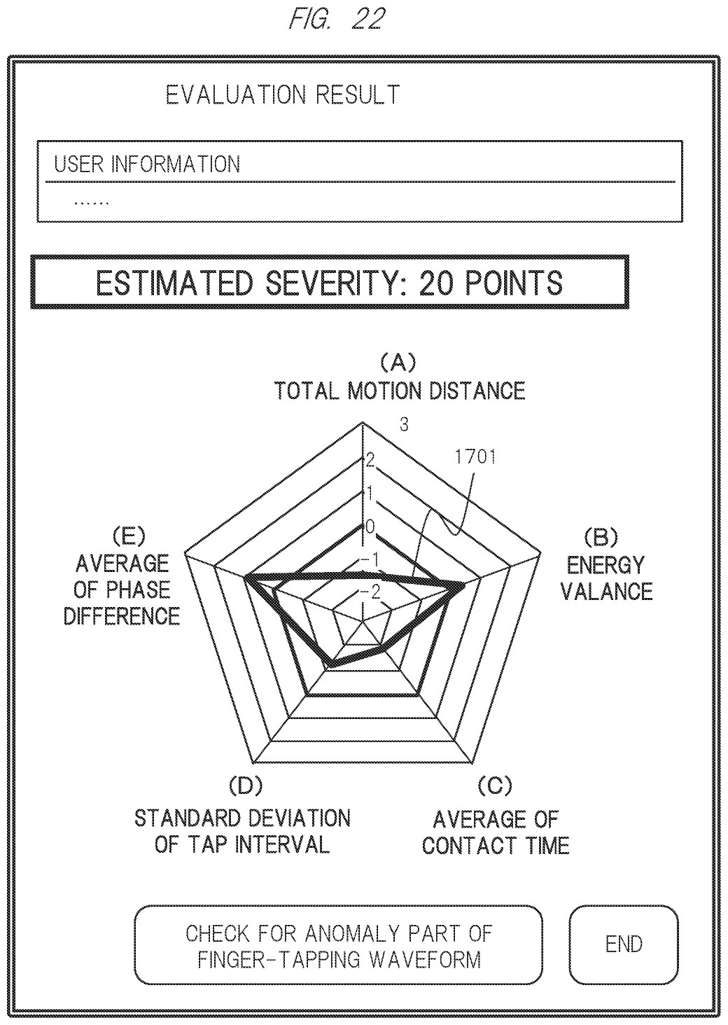

As still another example, FIG. 22 shows an evaluation result screen of the overall data. On this screen, analyzed evaluation result information of the task is displayed. This screen is automatically displayed after the analyzed evaluation of the task. This example shows a case of display of five feature amounts of A to E in the finger tapping motion to be displayed in a graph of a radar chart form. A frame line 1701 that is a solid line shows the analyzed evaluation result created after the present task measurement. An estimated severity score of the overall-data evaluation result 44C calculated by the overall-data evaluating unit 13B is displayed. A plurality of feature amounts are displayed in the radar chart form. In addition, an evaluation comment for the analyzed evaluation result or others may be displayed. The overall-data evaluating unit 13B creates the evaluation comment. For example, a message such as “(B) and (E) are good” is displayed. Inside the screen, operational buttons of “Confirmation for Anomaly Part of Finger-Tapping Waveform”, “End” and others are arranged. The cyclic time-series data anomaly-part detecting system 1 changes the screen to an anomaly-part detection result screen when the “Confirmation for Anomaly Part of Finger-Tapping Waveform” is selected, or changes it to the initial screen when the “End” is selected.

[Display Screen (4)—Anomaly-Part Detection Result]

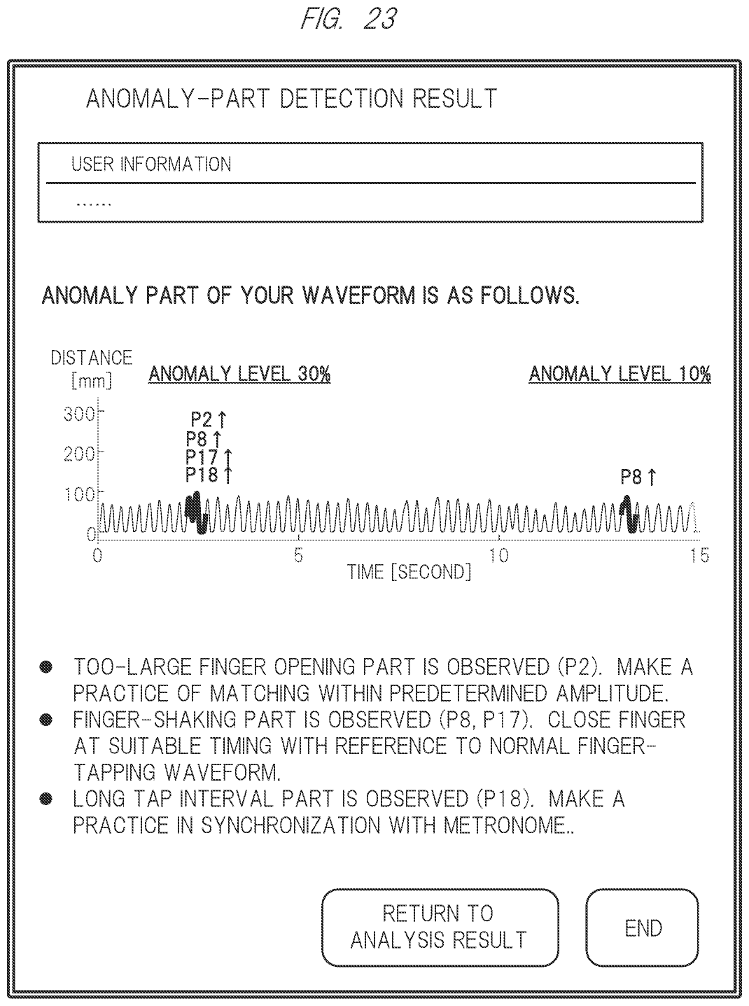

As still another example, FIG. 23 shows the anomaly-part detection result screen. On this screen, the partial-data anomaly detection result 46C calculated by the partial-data anomaly detecting unit 15C is shown to the user. A waveform of the overall data 44A is displayed with a thin line. The partial data 46A having the anomaly in the partial-data anomaly presence/absence 46Cb is displayed with a thick line on the waveform. Above this, the partial-data anomaly feature amount 46Cc and the partial-data anomaly level 46Ca are displayed. The partial-data anomaly feature amount 46Cc is denoted with an upward arrow when the feature-amount value is too large, or with a downward arrow when the feature-amount value is too small. The partial-data anomaly level 46Ca is displayed as the anomaly level. The evaluation comment for the partial-data anomaly feature amount 46Cc is also displayed. Further, the practice menu 47 for improving this is displayed.

The screen display method of the partial-data evaluation result shown in FIG. 23 is not limited to the graph of the time and the distance, and may be a graph of the time and the speed, the time and the acceleration or others. And, the display method is not limited to the graph display, and may be display in numerical-value data mode or a video display mode of the finger-tapping motion. In the case of the video display mode, a warning tone may be issued in the anomaly part, or a background of the video in the anomaly part may be changed so that the anomaly part can be recognized, and the display “P2, P8” or others may be displayed on a background screen.

[Display Screen (5)—Simultaneous Display of Overall-Data Evaluation Result and Anomaly-Part Detection Result]

It is more preferable to simultaneously display the screen of the overall-data evaluation result shown in FIG. 22 and the screen of the anomaly-part detection result shown in FIG. 23 on one screen. Since the overall-data evaluation result and the partial-data anomaly detection result match with each other, this case can provide effects of not losing the reliability of the system from the user, allowing a cause of the score of the overall-data evaluation result to be estimated through the screen of the anomaly-part detection result, and easily making the examinee understand or accept the cause of the score.

As the overall-data evaluation result in the simultaneous display of the content of the overall-data evaluation result and the content of the anomaly-part detection result, only the score may be displayed, only the radar chart may be displayed, both of the score and the radar chart may be displayed, or a different display method may be used. Similarly, as the anomaly-part detection result, the display is not limited to the graph display shown in FIG. 23, and a different display method can be used if the method causes a display mode in which the anomaly part of the overall data is visually recognized.

In the cyclic time-series data anomaly-part detecting system 1 of the first embodiment, the overall-data evaluating unit 13 detects the anomaly of the overall data 44A based on the overall-data feature amount 44B, and besides, creates the overall-data anomaly level 44Ca (the cyclic-information anomaly rate). The anomaly-part detecting system 1 creates the partial data 46A by dividing the overall data 44A that is the cyclic time-series data, calculates the partial-data feature amount 46B, and displays and outputs the partial-data anomaly detection result 46C that is the detection result of the anomaly of the partial data 46A, based on the partial-data feature amount 46B and the overall-data anomaly level 44Ca.

As described above, the anomaly-part detecting system 1 can avoid the results of the evaluation based on the overall data and the evaluation based on the partial data from being different from each other because of detecting the anomaly by using the overall-data anomaly level 44Ca for each piece of the partial data 46A resulted from the division of the overall data 44A.

In the cyclic time-series data anomaly-part detecting system 1 of the first embodiment, the overall-data evaluating unit 13 detects the anomaly of the overall data 44A based on the overall-data feature amount 44B, and besides, creates the feature-amount importance level 45B. The anomaly-part detecting system 1 creates the partial data 46A by dividing the overall data 44A that is the cyclic time-series data, calculates the partial-data feature amount 46B, and displays and outputs the partial-data anomaly detection result 46C that is the detection result of the anomaly of the partial data 46A, based on the partial-data feature amount 46B and the feature-amount importance level 45B.

As described above, the anomaly-part detecting system 1 can avoid the results of the evaluation based on the overall data and the evaluation based on the partial data from being different from each other because of detecting the anomaly by using the feature-amount importance level 45B for each piece of the partial data 46A resulted from the division of the overall data 44A.

The cyclic time-series data anomaly-part detecting system 1 of the first embodiment can detect the anomaly part of the overall data 44A and show it to the user by creating the partial data 46A by dividing the overall data 44A that is the cyclic time-series data, calculating the partial-data feature amount 46B, and providing the partial-data anomaly detection result 46C. In this case, the partial-data anomaly detection result 46C and the anomaly detection result of the overall data can be matched with each other by the application of the anomaly-rate determining unit 14A and the feature-amount importance-level determining unit 14B. When the overall-data evaluation result 44C is bad, the user can specifically recognize which part has the problem, from the partial-data anomaly detection result 46C. Further, since the practice menu 47 provided by the practice-menu determining unit 16 is shown, the user can recognize the practice method for improving the problem.

In the present embodiment, note that the anomaly-part detection targeting the time-series data of the finger-tapping motion has been explained. However, different data may be acceptable if the data is cyclic time-series data. For example, time-series data resulted from measurement of electrocardiographic signals, magnetocardiographic signals, pulse waves, breathing, brainwave, ambulation, eye blink, mastication and others are exemplified.

### Second Embodiment

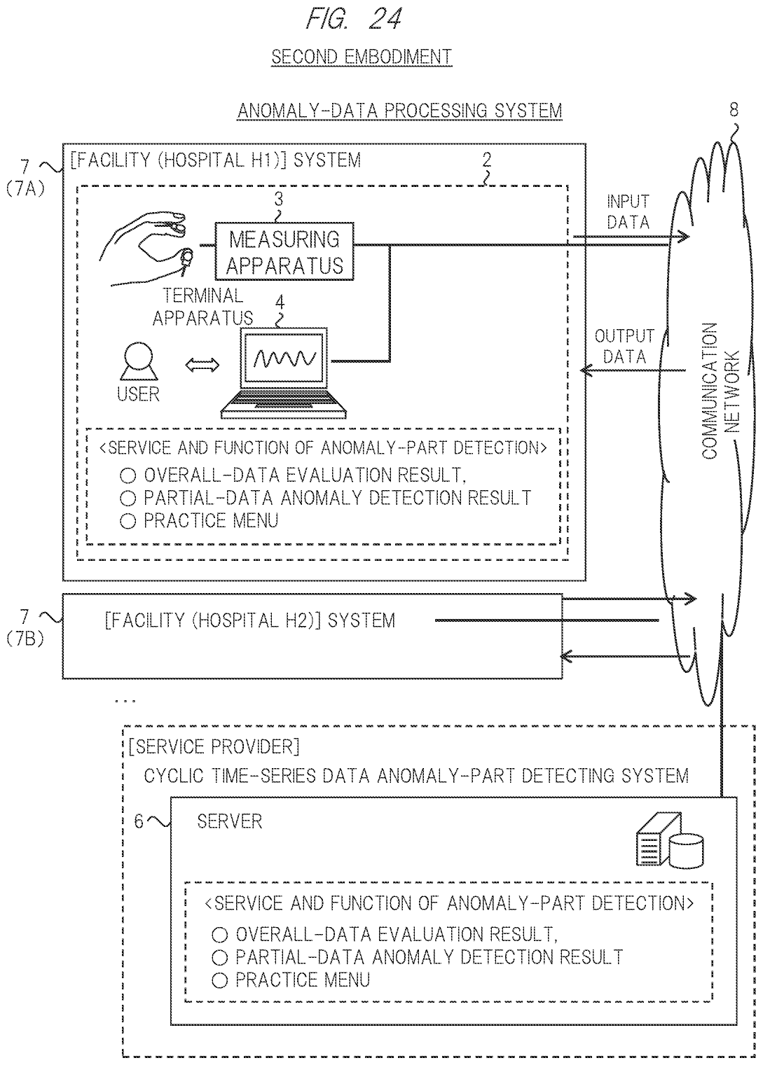

With reference to FIGS. 24 to 26, a cyclic time-series data anomaly-part detecting system of the second embodiment will be explained. A basic configuration of the second embodiment is the same as that of the first embodiment, and different parts of the configuration of the second embodiment from the configuration of the first embodiment will be explained.

[System]

FIG. 24 shows the cyclic time-series data anomaly-part detecting system of the second embodiment. The cyclic time-series data anomaly-part detecting system includes a server 6 of a service provider and systems 7 of a plurality of facilities, that are connected to each other by a communication network 8. The communication network 8 and the server 6 may include a cloud computing system.

As the facilities, various facilities such as a hospital, a health checking center, a public facility, an entertainment facility, and user's house are applicable. The system 7 is placed in the facility. As examples of the system 7 in the facility, a system 7A in a hospital H1, a system 7B in a hospital H2, and others are exemplified. Each of the system 7A and the system 7B in the respective hospitals includes the terminal apparatus 4 and the measuring apparatus 3 configuring the measuring system 2 that is the same as that of the first embodiment. A configuration of each system 7 may be the same or different. The system 7 in the facility may include an electronic-health-record managing system in the hospital or others. A measuring apparatus of the system 7 may be a dedicated device.

The server 6 is an apparatus managed by the service provider. The server 6 has a function of providing, to the facility and the user, the same partial-data anomaly detection service as that of the cyclic time-series data anomaly-part detecting system 1 of the first embodiment, as a service based on information process. The server 6 provides a service process in a client server system to the measuring system. The server 6 has a user managing function or others in addition to such a function. The user managing function is a managing function of registering and accumulating the user information of the user group, the measurement data, the analyzed evaluation data and others in the DB, the data and the information being provided from the systems in the plurality of facilities.

[Server]

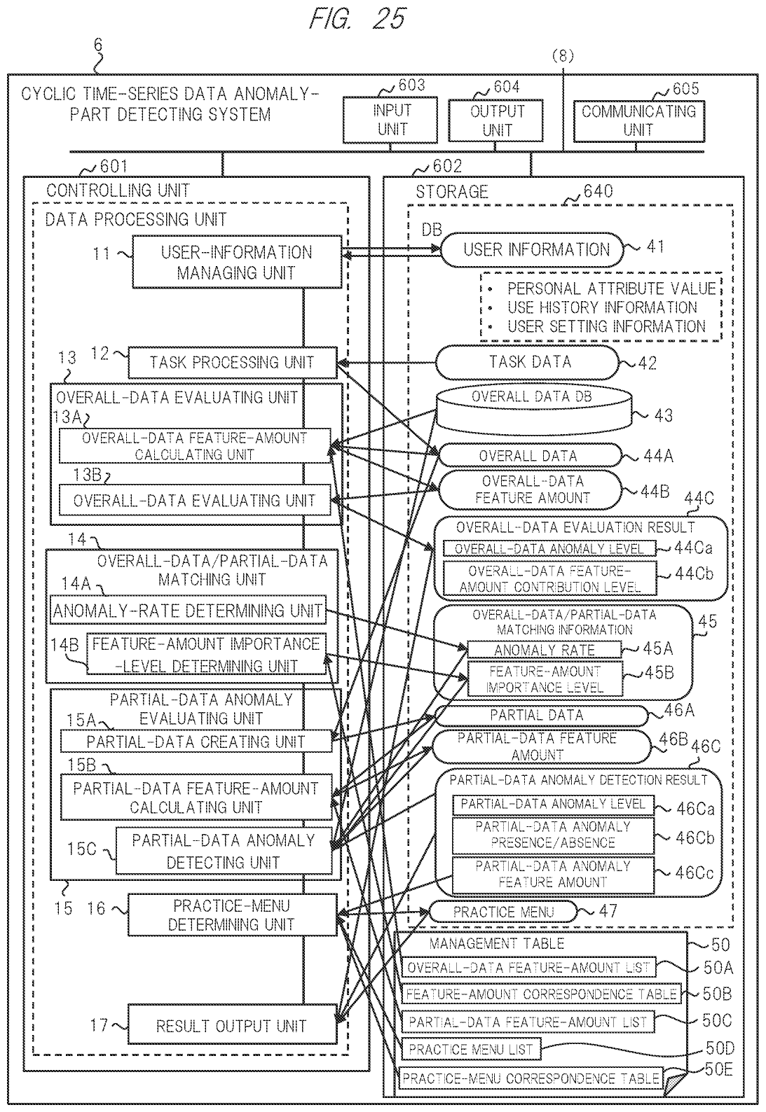

FIG. 25 shows a configuration of the server 6. The server 6 includes a control unit 601, a storage unit 602, an input unit 603, an output unit 604 and a communication unit 605, that are connected to each other by a bus. The input unit 603 is a unit performing the operational input made by the administrator of the server 6 or others. The output unit 604 is a unit performing the screen display to the administrator of the server 6 or others. The communication unit 605 is a unit including a communication interface and performing a communication process with the communication network 8. A DB 640 is built in the storage unit 602. The DB 640 may be managed by a DB server or others that is different from the server 6.

The control unit 601 controls the entire server 6, is made of a CPU, a ROM, a RAM and others, and achieves the data processing unit 600 detecting the anomaly data, determining the anomaly-data process or others, based on a software program process. The data processing unit 600 includes a user-information managing unit 11, a task processing unit 12, an overall-data evaluating unit 13, an overall-data/partial-data matching unit 14, a partial-data anomaly evaluating unit 15, a practice-menu determining unit 16 and a result output unit 17.

The user-information managing unit 11 registers and manages the user information related to the user group of the systems 7 in the plurality of facilities, as the user information 41 into the DB 640. The user information 41 includes an attribution value of each user, use history information, user setting information and others. The use history information includes actual-history information in which each user has used the anomaly-part detection service in past.

[Server Management Information]

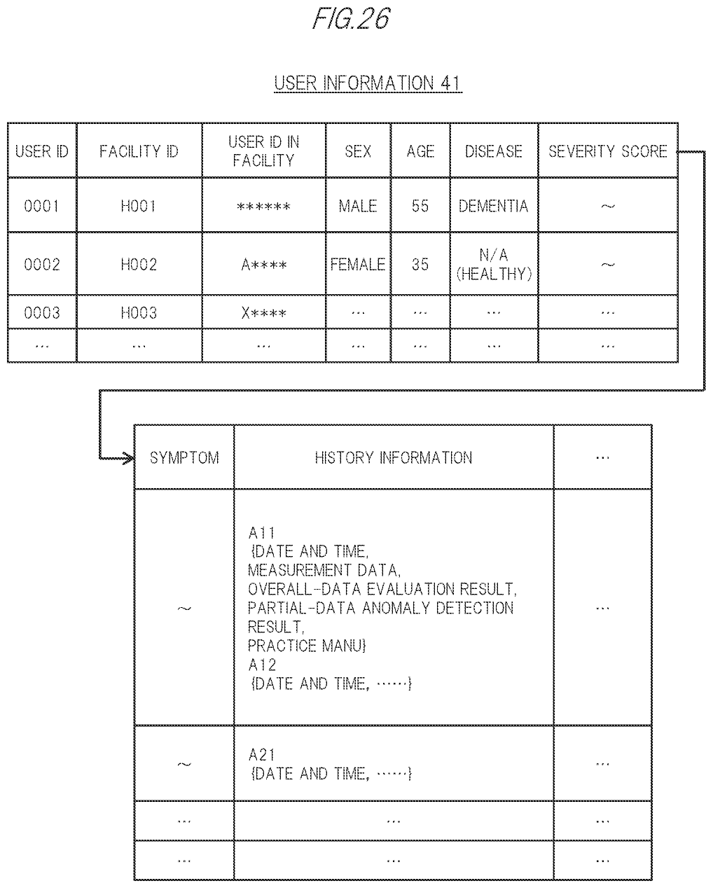

FIG. 26 shows a data configuration example of the user information 41 managed in the DB 640 by the server 6. A table for this user information 41 includes a user ID, a facility ID, a user ID in the facility, a sex, an age, a disease, a severity score, a symptom, history information and others. The user ID is optional identification information of the user in this system. The facility ID is identification information of the facility where the system 7 is placed. Note that a communication address of the measuring apparatus of each system 7 or others is also managed. The user ID in the facility is user identification information in a case with the user identification information managed in the facility or the system 7. In other words, the user ID and the user ID in the facility are corresponded to each other and managed. As the disease item or the symptom item, a value representing the disease or the symptom selected and input by the user or a value resulted from diagnosis of a doctor or others in the hospital is stored. The severity score is a value representing a level of the disease.

The history information item is information for managing the actual history of the user's usage of the anomaly-part detection service, and the information of date and time of each usage or others is stored in time series. As the history information item, data such as each data in the practice in this usage, that is the measurement data, the analyzed evaluation data, the anomaly-data detection result, the anomaly-data process content and others are stored. As the history information item, information of an address at which each data is stored may be stored.

[Effects and Others]

As similar to the first embodiment, the cyclic time-series data anomaly-part detecting system of the second embodiment can detect the anomaly part of the overall data 44A and show it to the user by creating the partial data 46A by dividing the overall data 44A that is the cyclic time-series data, calculating the partial-data feature amount 46B, and providing the partial-data anomaly detection result 46C. In this case, the partial-data anomaly detection result 46C and the anomaly detection result of the overall data can be matched with each other by the application of the anomaly-rate determining unit 14A and the feature-amount importance-level determining unit 14B. When the overall-data evaluation result 44C is bad, the user can specifically recognize which part has the problem, from the partial-data anomaly detection result 46C. Further, since the practice menu 47 provided by the practice-menu determining unit 16 is shown, the user can recognize the practice method for improving the problem.

In the foregoing, the present invention has been concretely described on the basis of the embodiments. However, the present invention is not limited to the foregoing embodiments, and various modifications can be made within the scope of the present invention.

The present invention is not limited to the embodiments, and includes various modification examples. For example, a part of the structure of one embodiment can be replaced with the structure of another embodiment, and besides, the structure of another embodiment can be added to the structure of one embodiment. Further, another structure can be added to/eliminated from/replaced with a part of the structure of each embodiment.

### EXPLANATION OF REFERENCE CHARACTERS

1 . . . cyclic time-series data anomaly-part detecting system, . . . measuring system, 3 . . . measuring apparatus, 4 . . . terminal apparatus

1. A detecting apparatus detecting anomaly by using cyclic information indicating a biological body state, comprising:
a cyclic-information acquiring unit configured to acquire the cyclic information;
a cyclic-information feature-amount calculating unit configured to calculate a feature amount of the cyclic information acquired by the cyclic-information acquiring unit;
a cyclic-information anomaly detecting unit configured to detect anomaly of the cyclic information based on the feature amount calculated by the cyclic-information feature-amount calculating unit;
an anomaly-rate creating unit configured to create a cyclic-information anomaly rate based on a result detected by the cyclic-information anomaly detecting unit;
a partial-information creating unit configured to create partial information based on the cycle by using the cyclic information acquired by the cyclic-information acquiring unit;
a partial-information feature-amount calculating unit configured to calculate a feature amount of the partial information created by the partial-information creating unit;
a partial-information anomaly detecting unit configured to detect anomaly of the partial information created by the partial-information creating unit, based on the feature amount calculated by the partial-information feature-amount calculating unit and the anomaly rate created by the anomaly-rate creating unit; and
an output unit configured to output information based on a result detected by the partial-information anomaly detecting unit and a result detected by the cyclic-information anomaly detecting unit.

2. The detecting apparatus according to claim 1,
wherein the partial-information anomaly detecting unit creates a level of the anomaly of the partial information created by the partial-information creating unit, information indicating whether the partial information created by the partial-information creating unit is anomalous or not, and information indicating an anomaly feature amount that is a feature amount to be a cause of the detection in which the partial information created by the partial-information creating unit is anomalous.

3. The detecting apparatus according to claim 2 further comprising:
a menu determining unit configured to determine a practice menu for improving the anomaly feature amount calculated by the partial-information anomaly detecting unit, and
wherein the output unit further outputs a menu determined by the menu determining unit.

4. A detecting apparatus detecting anomaly by using cyclic information indicating a biological body state, comprising:
a cyclic-information acquiring unit configured to acquire the cyclic information;
a cyclic-information feature-amount calculating unit configured to calculate a feature amount of the cyclic information acquired by the cyclic-information acquiring unit;
a cyclic-information anomaly detecting unit configured to detect anomaly of the cyclic information based on the feature amount calculated by the cyclic-information feature-amount calculating unit;
a feature-amount importance-level creating unit configured to create a feature-amount importance level based on a result detected by the cyclic-information anomaly detecting unit;
a partial-information feature-amount calculating unit configured to calculate a feature amount of the partial information created by the partial-information creating unit;
a partial-information anomaly detecting unit configured to detect anomaly of the partial information created by the partial-information creating unit, based on the feature amount calculated by the partial-information feature-amount calculating unit and the feature-amount importance level created by the feature-amount importance-level creating unit; and
an output unit configured to output information based on a result detected by the partial-information anomaly detecting unit and a result detected by the cyclic-information anomaly detecting unit.

5. The detecting apparatus according to claim 4,
wherein the output unit outputs screen information simultaneously showing a result detected by the partial-information anomaly detecting unit and a result detected by the cyclic-information anomaly detecting unit, on one screen.

6. A detecting method executed by a detecting apparatus detecting anomaly by using cyclic information indicating a biological body state, comprising:
a cyclic-information acquiring step of acquiring the cyclic information;
a cyclic-information feature-amount calculating step of calculating a feature amount of the cyclic information acquired in the cyclic-information acquiring step;
a cyclic-information anomaly detecting step of detecting anomaly of the cyclic information based on the feature amount calculated in the cyclic-information feature-amount calculating step;
an anomaly-rate creating step of creating a cyclic-information anomaly rate based on a result detected in the cyclic-information anomaly detecting step;
a partial-information creating step of creating partial information based on the cycle by using the cyclic information acquired in the cyclic-information acquiring step;
a partial-information feature-amount calculating step of calculating a feature amount of the partial information created in the partial-information creating step;
a partial-information anomaly detecting step of detecting anomaly of the partial information created in the partial-information creating step, based on the feature amount calculated in the partial-information feature-amount calculating step and the anomaly rate created in the anomaly-rate creating step; and
an output step of outputting information based on a result detected in the partial-information anomaly detecting step and a result detected in the cyclic-information anomaly detecting step.

7. A detecting method executed by a detecting apparatus detecting anomaly by using cyclic information indicating a biological body state, comprising:
a cyclic-information acquiring step of acquiring the cyclic information;
a cyclic-information feature-amount calculating step of calculating a feature amount of the cyclic information acquired in the cyclic-information acquiring step;
a cyclic-information anomaly detecting step of detecting anomaly of the cyclic information based on the feature amount calculated in the cyclic-information feature-amount calculating step;
a feature-amount importance-level creating step of creating a feature-amount importance level based on a result detected in the cyclic-information anomaly detecting step;
a partial-information creating step of creating partial information based on the cycle by using the cyclic information acquired in the cyclic-information acquiring step;
a partial-information feature-amount calculating step of calculating a feature amount of the partial information created in the partial-information creating step;
a partial-information anomaly detecting step of detecting anomaly of the partial information created in the partial-information creating step, based on the feature amount calculated in the partial-information feature-amount calculating step and the feature-amount importance level created in the feature-amount importance-level creating step; and
an output step of outputting information based on a result detected in the partial-information anomaly detecting step and a result detected in the cyclic-information anomaly detecting step.
